(19) United States
(12) Patent Application Publication
(10) Pub. No.: US 2023/0255832 A1
(43) Pub. Date: Aug. 17, 2023

(54) Stimulated Amniotic Cavity Biomimetic Dressing and Method Thereof

(71) Applicant: Zhongtang Wang, Xiamen (CN)

(72) Inventor: Zhongtang Wang, Xiamen (CN)

(21) Appl. No.: 18/111,835

(22) Filed: Feb. 20, 2023

(30) Foreign Application Priority Data
Feb. 17, 2022 (CN) 202210144331.7
Mar. 28, 2022 (CN) 202210310309.5

(51) Int. Cl. A61F 13/00

(52) U.S. Cl. CPC A61F 13/00068; A61F 2013/00089

(57) ABSTRACT

An intelligent bionic dressing (IBD) for simulating amniotic cavity includes an outer layer, an interlayer, an inner layer, a monitoring unit, a filling unit, a heating unit, a control unit, a waste liquid collection unit, and a plurality of accessories, configured to form a simulated amniotic cavity environment on a wound. Artificial amniotic fluid and its additives can circulate in the simulated amniotic cavity. The IBD provides a warm, moist, slightly acidic, sterile, pressureless and air isolated repair microenvironment for the wound, so as to monitor the wound in real-time, reduce dressing changes, rapid repair the wound, and reduce a scar.

### CROSS REFERENCE OF RELATED APPLICATION

This application is a non-provisional application that claims the benefit of priority under 35U.S.C.§119(e) to a Chinese application, application number 202210144331.7, filed Feb. 17, 2022, and a Chinese application, application number 202210310309.5, filed Mar. 28, 2022, which are incorporated herewith by reference in their entirety.

### BACKGROUND OF THE PRESENT INVENTION

### Field of Invention

The present invention relates to a field of medical materials, more particularly to a biomimetic dressing mimicking amniotic cavity and fabrication process and usage thereof.

### Description of Related Arts

The quality of wound healing could be affected by a variety of systemic and local factors. At the same time, it is particularly important to select appropriate wound dressings and reasonable dressing change measures.

New dressings such as negative pressure dressing, hydrogel dressing, aerogel dressing and intelligent dressing have significantly improved the quality of wound healing compared with traditional gauze dressing, but there are still many shortcomings such as unstable wound healing effect, dependence on medical experts for wound care, frequent dressing changes and inability to prevent scar hyperplasia.

Clinical studies have confirmed that a moist, oxygen free and slightly acidic environment is most suitable for human wound healing, and a fetal wound healing in maternal uterus is fast without obvious scar.

Therefore, simulating an amniotic cavity environment of fetal development in the maternal uterus, a bionic dressing with amniotic cavity function is constructed to provide a warm, moist, slightly acidic, sterile, zero pressure, air isolated microenvironment for human wounds, which is expected to achieve a clinical effect of reducing dressing changes, making wound management simple and easy, rapid wound repair, and insignificant scar hyperplasia.

### SUMMARY OF THE PRESENT INVENTION

The invention is advantageous in that it provides a bionic dressing simulating a function of amniotic cavity constructed and applied to a wound surface to form a warm, moist, slightly acidic, sterile, zero pressure, air isolated microenvironment in the wound area, so as to reduce dressing changes, simplify wound management, quickly repair the wound, and reduce scar growth.

Additional advantages and features of the invention will become apparent from the description which follows and may be realized by means of the instrumentalities and combinations particular point out in the appended claims.

According to the present invention, the foregoing and other objects and advantages are attained by an intelligent biomimetic dressing (IBD) according to a preferred embodiment of the application, which comprises an outer layer, an interlayer, an inner layer, a monitoring unit, a filling unit, a heating unit, a control unit, and a waste liquid collection unit. The outer layer is a transparent film structure on an outer surface of the IBD configured to isolate the wound surface from an outside air. The interlayer is arranged between the outer layer and the inner layer to provide a warm, humid, slightly acidic and sterile microenvironment, which comprises a microfluidic system. The inner layer is a porous transparent film structure on an inner surface of the IBD configured to directly cover the wound surface. The monitoring unit comprises at least one sensor, which is arranged on the interlayer, and is electrically connected with a power supply and the control unit for collecting a microenvironment data of the interlayer. The filling unit comprises a fluid reservoir, a first micropump, a micro valve and a connecting pipe, which is arranged at one end of the IBD, the connecting pipe is connected with the microfluidic system, the first micropump is electrically connected with the power supply and the control unit configured to add artificial amniotic fluid to the microfluidic system and provide a humid and slightly acidic environment for the interlayer. The heating unit comprises a heating component, which is arranged on the interlayer, the heating component is electrically connected with the power supply and the control unit configured to provide a 36.5° C. to 37.5° C. constant temperature environment for the interlayer. The control unit comprises a data storage processor, which is electrically connected with the intelligent terminal, power supply, sensor, micro pump, intelligent valve and heating component for data processing and controlling the micro pump, intelligent valve and heating component according to a program algorithm or manual remote control. The waste liquid collection unit comprises a connecting pipe, an intelligent valve, a second micropump, and a waste liquid capsule, the second micropump and intelligent valve are electrically connected with the power supply and control unit, the second micropump and intelligent valve are connected with the microfluidic system through connecting pipes to collect waste liquid from the interlayer and wound surface.

Further, the outer layer is made of transparent and soft medical polymer material or medical silica gel, with a thickness of 0.1 mm to 1.0 mm, preferably 0.2 mm to 0.5 mm. The outer layer’s circumference is beyond the interlayer from 5 mm to 25 mm, and a part of the outer layer beyond the interlayer is coated with adhesive.

Furthermore, the microfluidic system is a single-layer or multi-layer planar or three-dimensional structure. The microfluidic system comprises a first port, a capillary network, a plurality of second ports, and an end port. The first port passes through the outer layer and is connected with the fluid reservoir through a connecting pipe, a micro valve, and a micro pump. The first port is connected with the capillary network, and the second port is scattered on a wall of the capillary network. The capillary network is connected with the inner layer through the second port, and the end port passes through the outer layer and is connected with a waste liquid capsule through the connecting pipe and the intelligent valve.

Further, a capillary spacing of the capillary network is from 0.2 mm to 2.0 mm, preferably from 0.5 mm to 1.0 mm, an inner diameter of the capillary is from 0.1 mm to 0.8 mm, preferably from 0.2 mm to 0.5 mm, a wall thickness of the capillary is from 0.01 mm to 1.0 mm, preferably from 0.05 mm to 0.25 mm, a spacing of the second port on the capillary is from 0.5 mm to 1.0 mm with an aperture from 0.02 mm to 0.2 mm.

Further, the capillary is fabricated with a transparent and soft medical polymer material or medical silica gel with a coating on an outer wall of the capillary, the coating includes a bacteriostatic material.

Further, the sensors comprise a combination of at least one or more of a temperature sensor, a humidity sensor, an oxygen saturation sensor, a pressure sensor, a pH sensor, and a microbial sensor.

Further, the reservoir comprises an injection pot for connecting a syringe needle, a capsular bag for storing artificial amniotic fluid which includes a buffer. When the pH sensor detects an intralayer microenvironment above pH 6.0 or below pH 4.0, the buffer can be passed through the connecting tube, the first port, and the second port permeate into the inner layer so that the pH of a wound environment can maintained between pH 6.0 and pH 4.0.

Further, the reservoir can be configured to store an antibiotic or pain relief agent or a lytic enzyme or growth factor or stem cell, after receiving instructions, the micropump is initiated and the antibiotic or pain relief agent or lytic enzyme or growth factor or stem cell inside the reservoir can be infiltrated into the inner layer through the connecting tube, the first port, the capillary network, the second ports for wound treatment.

Further, the intralayer further comprises at least one lamp, which emits infrared and/or far infrared and / or ultraviolet light for wound treatment.

Preferably, the inner layer can be a frame structure placed around the wound surface configured to separate the intralayer from direct contact with the wound surface, and eliminate wound interference by the IBD.

Further, the control unit includes a wireless communication component for transmitting monitoring data and receiving instructions, or transmitting data to a server or mobile smart terminal, establishing a wireless communication.

On the other hand, an embodiment of the present application provides a fabrication method of an IBD, which comprises at least one step as follows: planning and designing the IBD, preparing an outer layer, preparing a intra layer which including designing and making a microfluidic systems, laying down a monitoring unit, a heating unit, and a plurality of lamps, preparing an inner layer, preparing a filling unit which including designing and making a plurality of reservoirs, a plurality of micropumps, a plurality of microvalves, a plurality of connecting or extending tubes, and a multi-pass tube, preparing a control unit and a power supply, preparing a waste liquid collection unit which including designing and making an intelligent valve and a reservoirs, assembling the IBD which including, but is not limited to: (1) combining the outer layer, the intralayer and the inner layer, (2) pluggable connecting the microfluidic system with the filling unit, (3) pluggable connecting the microfluidic system with the waste liquid collecting unit, (4) pluggable connecting the filling unit, the waste liquid collecting unit, the heating unit, the connecting tubes and the monitoring unit with the power supply and the control unit, and then encapsulating and sterilizing.

On the other hand, an embodiment of the present application provides a usage method of an IBD, which comprises at least one step as follows: selecting an IBD or personalized tailoring according to a wound type and size, preparing the wound, wound dressing with the IBD, configuring an artificial amniotic fluid, a buffer and additives and then injecting into a reservoir, setting up an intelligent control program or artificial intervention program, starring, monitoring data real-time to provide a warm, moist, slightly acidic, isolated, unstressed, sterile repair microenvironment for wound, when wound healing, removing the IBD.

Further, the usage method of the IBD of an embodiment of the present application includes at least one step as follows: planning the IBD, and configurating a plurality of functional units according to the wound type and size, clipping an outer layer and inner layer bedside, selecting one or more microfluidic systems, bonding or datable connecting sensors, heating components, connecting tubes to make a intralayer, preparing a wound, covering the wound with the inner layer, the intralayer, the outer layer, and the microfluidic system which being pluggable connecting with a filling unit, a waste liquid collection unit, a power supply, a monitoring unit, a heating unit, a lamp, a control unit, and a waste liquid collection unit, configurating an artificial amniotic fluid and additives, and injecting into a reservoir, setting up an intelligent control program or artificial intervention program, then starring, monitoring data real-time, when the wound healing, removing the IBD.

On the other hand, an embodiment of the present application provides a simulated amniotic cavity biomimetic dressing configured for providing a closed, moist, slightly acidic, sterile healing microenvironment to a wound bed, which includes a dressing main body, and a plurality of dressing fittings, the dressing main body comprises a cover film, a frame, a fixation membrane, the cover film can cover the frame so as to form a closed chamber with the wound bed, the fixation membrane can encircle the framework and fix a skin. The framework includes a hollow canal configured to circulating artificial amniotic fluid. The dressing fittings comprise a tube line, an injection pot or valve configured to inject and/or discharge artificial amniotic fluid.

Further, the cover film is a transparent film integrated with a lateral anchorage on the framework.

Further, the fixation membrane is covered with a back adhesive, with a width more than 10 mm, preferably from 15 mm to 25 mm, integrated with a lower lateral edge of the framework.

Further, the framework, the fixation membrane and the cover film can be combined an integrated structure that covers the wound to form a closed chamber.

Preferably, according to the wound style and size, the framework, the fixation membrane and the cover film can be cut and ped and combined an integrated structure to form a closed chamber for the wound.

Further, an IBD includes a dressing attachment, the dressing attachment comprises a display which can be set on an inside of a framework, the display comprises at least one or more of a pH display unit, a temperature display unit, a humidity display unit, and a microbial display unit.

Preferably, the pH display unit comprises a micro pH detection rod which includes an indicator core and a transparent shell, the indicator core comprises a methyl red indicator and a fiber rod, the transparent shell comprises a plurality of micro pores.

Preferably, the temperature display unit comprises a thermometer.

Preferably, the humidity display unit comprises a hygrometer.

Preferably, the microorganism display unit comprises a micro microorganism detection cassette which includes a plurality of micro test strips arranged in parallel or serially within a transparent cassette, the micro detection strip comprises at least one or more combinations of a gram negative bacteria test strip, a gram positive bacteria test strip, a fungi test strip, respectively.

Further, the hollow canal can be set either inside or outside a framework, with a lumen conduit diameter of no more than 5 mm, preferably from 0.5 mm to 2.5 mm.

Further, the hollow canal comprises a plurality of micropores, with a diameter no more than 0.5 mm, preferably from 0.05 mm to 0.25 mm and a space between micropores no more than 20 mm, preferably from 5 mm to 10 mm.

Further, the hollow canal comprises at least one side hole, with an aperture no more than 2.5 mm, preferably from 0.5 mm to 1.0 mm.

Further, the tube line comprise an inlet tube and an outlet tube configured to inject artificial amniotic fluid into the hollow canal and drain artificial amniotic fluid from the hollow canal or closed chamber respectively.

On the other hand, an embodiment of the present application provides a usage method of a simulated amniotic cavity biomimetic dressing, which comprises steps of:
(1) selecting a fabricated simulated amniotic cavity biomimetic dressing;
(2) pasting and fixing the fabricated simulated amniotic cavity biomimetic dressing on a wound;
(3) adding artificial amniotic fluid and additives;
(4) observing the wound through the cover film, checking the display, refilling or draining artificial amniotic fluid if necessary; and
(5) removing the fabricated simulated amniotic cavity biomimetic dressing when wound healed.

Further, in the step (1), a simulated amniotic cavity biomimetic dressing can also be fabricated bedside including:
(a) evaluating wound shape and size;
(b) cropping a cover film, a fixation membrane and framework according to the wound shape and size;
(c) combining the cover film, the fixation membrane and the framework as integrated structure;
(d) bonding a display with the framework; and
(e) connecting a dressing fitting.

### Beneficial Effect

(1) The IBD is thin, light, flexible, and transparent, so that a whole process of wound healing can be in a warm, moist, slightly acidic, sterile, pressure free, and closed microenvironment similar to that of a fetus in mother uterus, thus obtaining clinical effects of accelerated wound healing with insignificant scarring.

(2) The IBD can provide digitalized clinical evidence for smart decision-making or artificial intervention through monitoring dynamic changes of wound environment, such as temperature, humidity, acid-base, wound pressure, wound microcirculation, bacteria, etc.

(3) The IBD can intelligently control the operation of the filling unit, heating unit, lamps, waste liquid collection unit to optimize the local microenvironment of the wound according to a program algorithm real-time.

(4) the artificial amniotic fluid can be flexibly configured according to wound characteristics and healing stages, such as wound pain with anesthetic agents, chronic difficult healing wounds with growth factors or stem cells, residual wounds with necrotic tissue with lytic enzymes, suspicious infected wounds with sensitive antibiotic agents, moreover, because a thickness of the IBD is less than 1 mm, so that the demand of artificial amniotic fluid is minimal, subsequently reduce a waste of medicines.

(5) the fill unit, the waste liquid collection unit, and the heating unit or sensors can be recycled and reused, thus reducing wound healing cost.

(6) The inner layer, intralayer and outer layer can be suitably shaped when used in order to apply on a wound. In addition, the inner layer only included a framework so that the wound was in zero contact with the IBD, achieving an effect of zero disturbing the wound.

(7) there is essentially no need for dressing change, the wound situation can be directly observed and can be intelligently or remotely managed, which simplifies the wound care process and reduces a burden of care.

(8) The simulated amniotic cavity biomimetic dressing of the present application is simple in structure and can achieve a biomimetic effect of simulated amniotic cavity without the need for a complex and highly refined microfluidic system and its micropump microvalve components.

(10) The simulated amniotic cavity biomimetic dressing of the present application requires no additional power supply and is both weight-saving and environmentally friendly.

(11) The simulated amniotic cavity biomimetic dressing in this application is simple and easy to fill and drain artificial amniotic fluid with a syringe injection. When continuous irrigation with artificial amniotic fluid is required for wound, a most common instrument can be chosen, such as inlet connection infusion bag instillation and outlet connection collecting bag drainage.

(12) The simulated amniotic cavity biomimetic dressing and its component materials of the application are easy to obtain, low in cost, and can be manufactured in batches, which increases the accessibility of ordinary patients to this advanced bionic dressing

These and other objectives, features, and advantages of the present invention will become apparent from the following detailed description, the accompanying drawings, and the appended claims.

### BRIEF DESCRIPTION OF THE DRAWINGS

In order to more clearly explain the technical solution of the embodiments of the present application, the following will briefly introduce the drawings needed to be used in the description of the embodiments. Obviously, the drawings in the following description are only some embodiments of the present application. For those skilled in the art, other drawings can be obtained from these drawings without any creative effort.

In addition, the drawings are only schematic diagrams of the application and are not necessarily drawn to scale. The same reference numerals in the figures represent the same or similar parts, and therefore repeated description of them will be omitted. Some block diagrams shown in the figures are functional entities, which do not necessarily correspond to physically or logically independent entities. These functional entities can be implemented in one or more hardware modules or component combinations.

FIG. 1 is a schematic view illustrating a structure of a front view of a biomimetic dressing according to a preferred embodiment of the present application.

FIG. 2 is a schematic view illustrating a structure of a back surface architecture of a biomimetic dressing according to the above preferred embodiment of the present application.

FIG. 3 is a schematic view illustrating another structure of a back surface architecture of a biomimetic dressing according to the above preferred embodiment of the present application.

FIG. 4 is a schematic view illustrating a structure of a front view of an interlayer according to the above preferred embodiment of the present application.

FIG. 5 is a schematic view illustrating a structure of a combinatorial connectivity of an intelligent biomimetic control part according to the above preferred embodiment of the present application.

FIG. 6 is a schematic view illustrating a structure of an IBD comprising two inner layers, an interlayer, and an outer layer according to the above preferred embodiment of the present application.

FIG. 7A is a schematic view illustrating a structure of an outer back surface of an outer layer according to the above preferred embodiment of the present application.

FIG. 7B is a schematic view illustrating a first structure of an outer layer cross-sectional structure according to the above preferred embodiment of the present application.

FIG. 7C is a schematic view illustrating a second structure of an outer layer cross-sectional structure according to the above preferred embodiment of the present application.

FIG. 7D is a schematic view illustrating a third structure of an outer layer cross-sectional structure according to the above preferred embodiment of the present application.

FIG. 7E is a schematic view illustrating a fourth structure of an outer layer cross-sectional structure according to the above preferred embodiment of the present application.

FIG. 7F is a fifth schematic structure of an outer layer cross-sectional structure according to the above preferred embodiment of the present application.

FIG. 8A is a schematic view illustrating a first structure of an inner layer according to the above preferred embodiment of the present application.

FIG. 8B is a schematic view illustrating a second structure of an inner layer according to the above preferred embodiment of the present application.

FIG. 8C is a schematic view illustrating a third structure of an inner layer according to the above preferred embodiment of the present application.

FIG. 8D is a schematic view illustrating a fourth structure of an inner layer according to the above preferred embodiment of the present application.

FIG. 9A is a schematic view illustrating a first structure of a microfluidic system according to the above preferred embodiment of the present application.

FIG. 9B is a schematic view illustrating a second structure of a microfluidic system according to the above preferred embodiment of the present application.

FIG. 9C a schematic view illustrating is a third structure of a microfluidic system according to the above preferred embodiment of the present application.

FIG. 9D is a schematic view illustrating a fourth structure of a microfluidic system according to the above preferred embodiment of the present application.

FIG. 9E is a schematic view illustrating a fifth structure of a microfluidic system according to the above preferred embodiment of the present application.

FIG. 9F is a schematic view illustrating a sixth structure of a microfluidic system according to the above preferred embodiment of the present application.

FIG. 9G is a schematic view illustrating a seventh structure of a microfluidic system according to the above preferred embodiment of the present application.

FIG. 9H is a schematic view illustrating an eighth structure of a microfluidic system according to the above preferred embodiment of the present application.

FIG. 10 is a schematic view illustrating a structure of an interlayer with a heating part according to the above preferred embodiment of the present application.

FIG. 11 is a schematic view illustrating a structure of an interlayer with a lamp tube according to the above preferred embodiment of the present application.

FIG. 12 is a schematic view illustrating a structure of an interlayer with a temperature sensor according to the above preferred embodiment of the present application.

FIG. 13 is a schematic view illustrating a structure of an interlayer with a pH sensor according to the above preferred embodiment of the present application.

FIG. 14 is a schematic view illustrating a structure of an interlayer with a humidity sensor according to the above preferred embodiment of the present application.

FIG. 15 is a schematic view illustrating a structure of an interlayer with a microbial sensor according to the above preferred embodiment of the present application.

FIG. 16 is a schematic view illustrating a structure of an interlayer with an oxygen saturation sensor according to the above preferred embodiment of the present application.

FIG. 17 is a schematic view illustrating a structure of an interlayer with a pressure sensor according to the above preferred embodiment of the present application.

FIG. 18 is a schematic view illustrating a structure of an interlayer with an integrated sensors according to the above preferred embodiment of the present application.

FIG. 19A a schematic view illustrating a structure of filling unit according to the above preferred embodiment of the present application.

FIG. 19B a schematic view illustrating another structure of filling unit according to the above preferred embodiment of the present application.

FIG. 20A a schematic view illustrating a a structure of waste liquid collection unit according to the above preferred embodiment of the present application.

FIG. 20B is a schematic view illustrating another structure of waste liquid collection unit according to the above preferred embodiment of the present application.

FIG. 21 is a schematic view illustrating a structure of a control according to the above preferred embodiment of the present application.

FIG. 22 is a schematic view illustrating a structure of a frontal view of a simulated amniotic cavity biomimetic dressing according to the above preferred embodiment of the present application.

FIG. 23 is a schematic view illustrating a second structure of a frontal view of a simulated amniotic cavity biomimetic dressing according to the above preferred embodiment of the present application.

FIG. 24 is a schematic view illustrating a third structure of a frontal view of a simulated amniotic cavity biomimetic dressing according to the above preferred embodiment of the present application.

FIG. 25 is a schematic view illustrating a fourth structure of a frontal view of a simulated amniotic cavity biomimetic dressing according to the above preferred embodiment of the present application.

FIG. 26 is a schematic view illustrating a structure of a back view of a simulated amniotic cavity biomimetic dressing according to the above preferred embodiment of the present application.

FIG. 27A is a schematic view illustrating a structure of a framework of a simulated amniotic cavity biomimetic dressing according to the above preferred embodiment of the present application.

FIG. 27B is a schematic view illustrating another structure of a framework of a simulated amniotic cavity biomimetic dressing according to the above preferred embodiment of the present application.

FIG. 28A is a schematic view illustrating a structure of a cross-sectional view of a framework of a simulated amniotic cavity biomimetic dressing according to the above preferred embodiment of the present application.

FIG. 28B is a schematic view illustrating a second structure of a cross-sectional view of a framework of a simulated amniotic cavity biomimetic dressing according to the above preferred embodiment of the present application.

FIG. 28C is a schematic view illustrating a third structure of a cross-sectional view of a framework of a simulated amniotic cavity biomimetic dressing according to the above preferred embodiment of the present application.

FIG. 28D is a schematic view illustrating a fourth structure of a cross-sectional view of a framework of a simulated amniotic cavity biomimetic dressing according to the above preferred embodiment of the present application.

FIG. 29A is a schematic view illustrating a connective structure a framework and a connecting tube of a simulated amniotic cavity biomimetic dressing according to an example of the present application;

FIG. 29B is another schematic view illustrating another structure of a connective structure a framework and a connecting tube of a simulated amniotic cavity biomimetic dressing according to the above preferred embodiment of the present application.

FIG. 30 is a schematic view illustrating a structure of a framework and a display component of a simulated amniotic cavity biomimetic dressing according to the above preferred embodiment of the present application.

FIG. 31A is a schematic view illustrating a structure of a framework and an indication unit of a simulated amniotic cavity biomimetic dressing according to the above preferred embodiment of the present application.

FIG. 31B is a schematic view illustrating a second structure of a framework and an indication unit of a simulated amniotic cavity biomimetic dressing according to the above preferred embodiment of the present application.

FIG. 31C is a schematic view illustrating a third structure of a framework and an indication unit of a simulated amniotic cavity biomimetic dressing according to the above preferred embodiment of the present application.

FIG. 31D is a schematic view illustrating a fourth structure of a framework and an indication unit of a simulated amniotic cavity biomimetic dressing according to the above preferred embodiment of the present application.

FIG. 32 is a schematic view illustrating a connection structure of a simulated amniotic cavity biomimetic dressing for a wound according to the above preferred embodiment of the present application.

FIG. 33 is a schematic view illustrating a connected structure of a simulated amniotic cavity biomimetic dressing for wound manipulation according to the above preferred embodiment of the present application.

FIG. 34 is a schematic view illustrating a second connected structure of a simulated amniotic cavity biomimetic dressing for wound manipulation according to the above preferred embodiment of the present application.

FIG. 35 is a schematic view illustrating a third connected structure of a simulated amniotic cavity biomimetic dressing for wound manipulation according to the above preferred embodiment of the present application.

The drawings, described above, are provided for purposes of illustration, and not of limitation, of the aspects and features of various examples of embodiments of the invention described herein. The drawings are not intended to limit the scope of the claimed invention in any aspect. For simplicity and clarity of illustration, elements shown in the drawings have not necessarily been drawn to scale and the dimensions of some of the elements may be exaggerated relative to other elements for clarity.

### DETAILED DESCRIPTION OF THE PREFERRED EMBODIMENT

In order to make the purpose, technical solution and advantages of the application more clearly, the application is further described in detail below in combination with embodiments. It should be understood that the specific embodiments described herein are only used to explain the application, not to limit the application.

It should be noted that the upper, lower, left, right, far, near, front and rear directions in this embodiment are only relative concepts to each other or refer to the normal use state of the product, and should not be considered as restrictive.

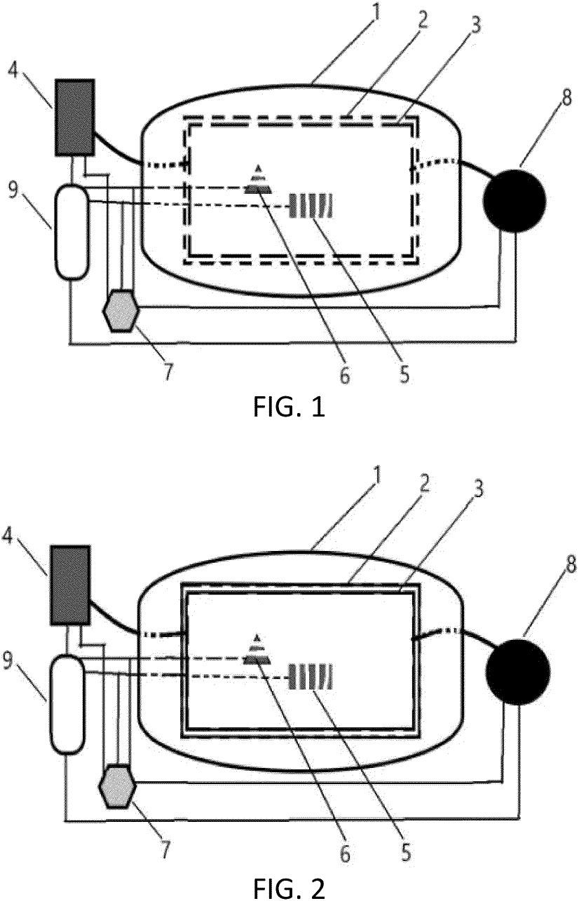

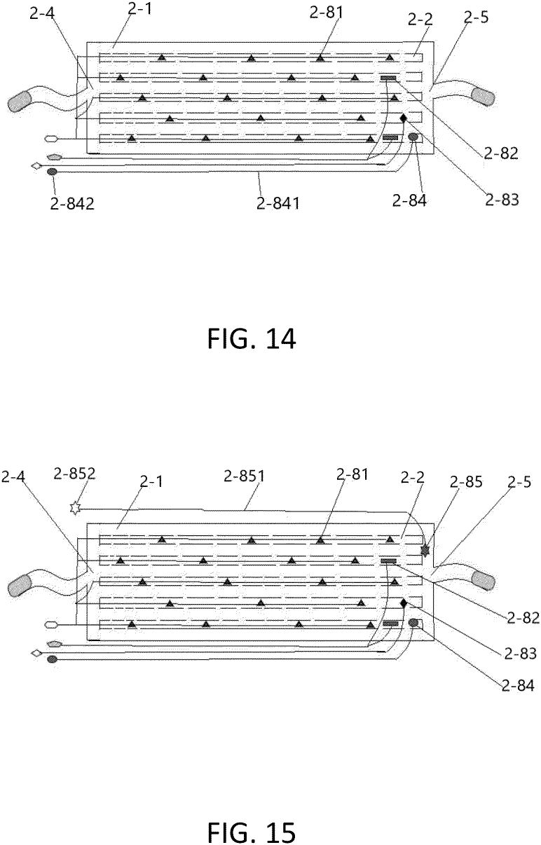

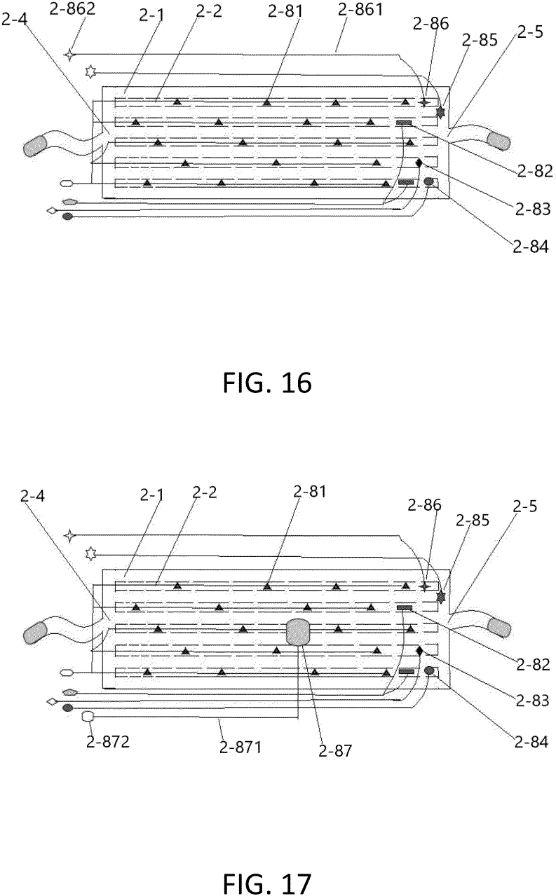

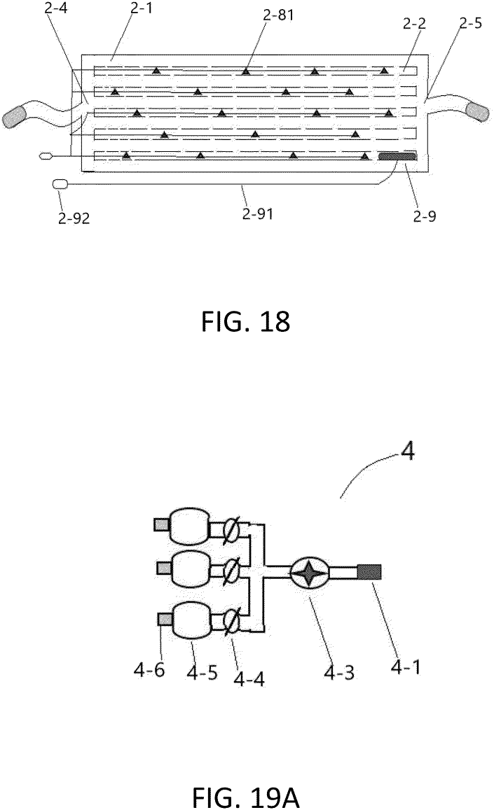

Referring to FIGS. 1-21, an IBD (intelligent biomimetic dressing) according to a preferred embodiment of the present invention is illustrated, wherein the IBD comprises an outer layer 1, an interlayer 2, an inner layer 3, a filling unit 4, a monitoring unit 5, a heating unit 6, a lamp tube 2-88, a power supply 7, a waste liquid collection unit 8, a control unit 9, and a patch film 10.

As shown in FIGS. 1 and 2, the IBD according to the preferred embodiment of the present invention comprises an outer layer 1, an interlayer 2, an inner layer 3, a filling unit 4, a monitoring unit 5, a heating unit 6, a lamp tube 2-88, a power supply 7, a waste liquid collection unit 8, and a control unit 9. The outer layer 1 can be set up on an outer surface of the IBD, the outer layer 1 are beyond a dimension of the interlayer 2 and inner layer 3 in all angle sizes, and when the outer layer 1 is pasted with a healthy skin around a wound, the outer layer 1 can ensure that the interlayer 2 and the inner layer 3 are completely sealed from an outside air, so that the wound is in an anaerobic or hypooxic state. The inner layer 3 can be set up on an inner surface of the smart biomimetic dressing for directly covering the wound surface, or upper the wound so as to avoid direct contact with the wound. The s interlayer 2 can be set between the outer layer 1 and the inner layer 3, the interlayer 2 main body structure can include a microfluidic system, when the filling unit 4 performs the control unit 9 instruction, artificial amniotic fluid can infiltrate the wound through the microfluidic system. The monitoring unit 5 and the heating unit 6 can be set between the microfluidic system gap 2-2, the monitoring unit 5 can real-time collect the wound surface temperature, humidity, pH, and microorganisms and transmit data to the control unit 9, the control unit 9 can process the data and issue instructions, the filling unit 4 or heating unit 6 receive the instructions, adjust the filling unit 4 or heating unit 6 work procedures in real time, change the artificial amniotic fluid temperature or humidity or pH value of the wound surface or clear microorganisms, so that the wound surface always maintains a warm, moist, slightly acid, sterile microenvironment. The filling unit 4 can be exteriorized, and can be connected with the microfluidic system for the infusion of artificial amniotic fluid into the microfluidic system. The filling unit 4 can also be electrically connected with the power supply 7 and the control unit 9, and of course, a part of the filling unit 4 can also be set up inside the interlayer 2. The power supply 7 can use batteries, or an exogenous supply device to supply energy for the filling unit 4, the monitoring unit 5, the heating unit 6, the waste liquid collection unit 8, the control unit 9, the lamp tube 2-88, if the power supply 7 is a battery, it can be set outside or it can be set inside the interlayer 2. The waste liquid collection unit 8 can be exteriorized and set at an end side of the outer layer 1, which can be connected with the interlayer 2 microfluidic system, and the power supply 7 and the control unit 9, which are used to pool waste liquid from the wound and microfluidic system.

In addition, the IBD exotypes and sizes can be set to arbitrary exotypes and sizes on demand, which can be either planar 2D or 3D stereo-structure. In addition, the interlayer 2 and inner layer 3 dimensions are generally consistent, and the interlayer 2 dimension can also extend slightly beyond the inner layer 3. In addition, the filling unit 4, the power supply 7 and the control unit 9 can be set separately, and two or three components of the filling unit 4 and / or the power supply 7 and / or the control unit 9 can also be combined into an integrated structure.

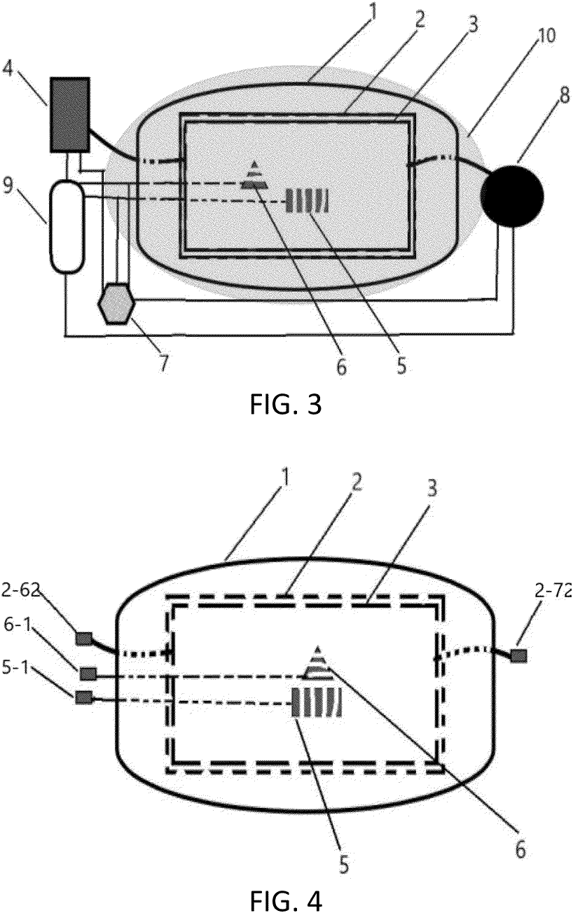

As shown in FIG. 3, according to the preferred embodiment of the present application, the IBD may further comprise a patch membrane 10. The patch membrane 10 can be relatively taped to the outer layer 1, protecting the interlayer 2 and the inner layer 3. The patch membrane 10 can be made in medical plastic with dimensions slightly beyond the outer layer 1 that can bond to the outer layer 1 edge. In addition, the patch membrane 10 can also be replaced by an absorbent shaped encapsulating box to achieve temporary fixation of the outer layer 1, the inter layer 2 and the inner layer 3 as well as to avoid contamination effects.

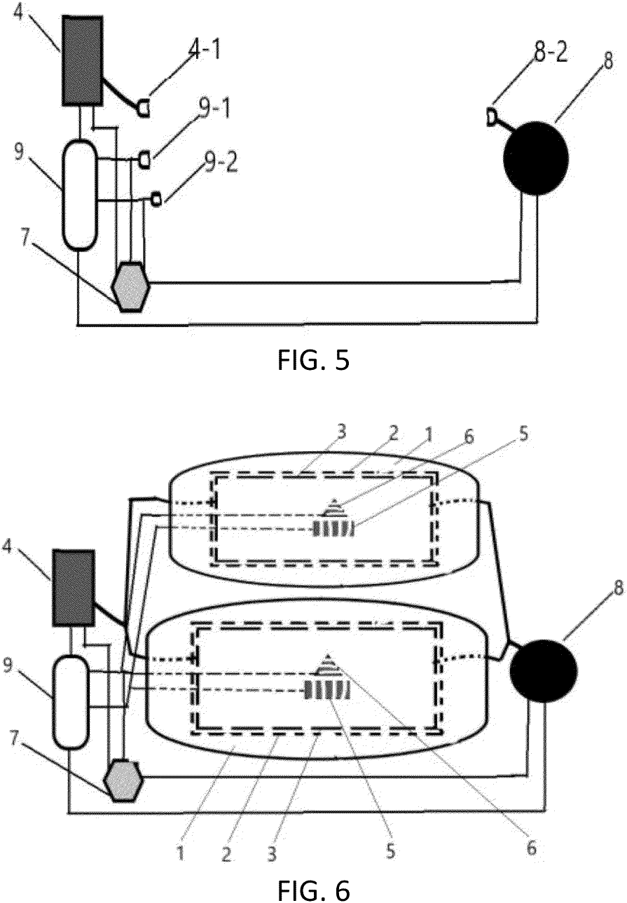

Referring to FIGS. 4 and 5, according to the preferred embodiment of the present application, the IBD can be divided into two relatively separate parts. As shown in FIG. 4, the outer layer 1, the inter layer 2, the inner layer 3 of one IBD, as well as the monitoring unit 5 and the heating unit 6 attached to the inter layer 2 can be combined independently. As shown in FIG. 5, the filling unit 4, the power supply 7, the waste liquid collection unit 8, the control unit 9 of one IBD of the application can be combined independently. When used, a first interface 2-62 and a second interface 2-72 of the microfluidic system can be connected pluggable with a third interface 4-1 of the filling unit 4, and a fourth interface 8-2 of the waste liquid collection unit 8, respectively, and a fifth interface 5-1 of the monitoring unit 5 and a sixth interface 6-1 of the heating unit 6 can be connected with a seventh interface 9-1 of the power supply 7 and an eighth interface 9-2 of the control unit 9, respectively, so that a fully functional IBD can be assembled.

As shown in FIGS. 4 and 5, relatively separate two parts of the IBDs can be assembled into fully functional IBDs as shown in FIGS. 1 to 3, or as shown in FIG. 6 which is composed of two relatively separate parts containing the outer layer 1, the inner layer 3, and the inter layer 2 with the heating unit 6 and the monitoring unit 5 combined with a set of attached components containing the filling unit 4, the power supply 7, the waste liquid collection unit 8, and the control unit 9 being assembled to a complete functional IBDs. Moreover, three or more sets of relatively independent parts containing the outer layer 1, the inner layer 3, and the inter layer 2 with the heating unit 6 and the monitoring unit 5 combined with a set of attached components containing the filling unit 4, the power supply 7, the waste liquid collection unit 8, and the control unit 9 can be assembled to a complete functional IBD for multiple independent wound treatment as required, so that the multi sets containing the outer layer 1, inner layer 3, and the inter layer 2 attached with the monitoring unit 5, the heating unit 6 combine to share a set component containing the filling unit 4, the power supply 7, the waste liquid collection unit 8, and the control unit 9 combination to reduce the cost of multiple independent wound treatment.

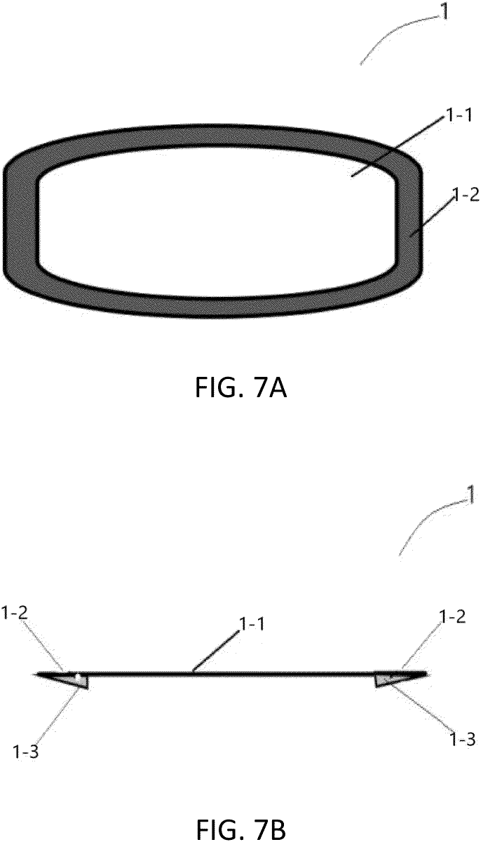

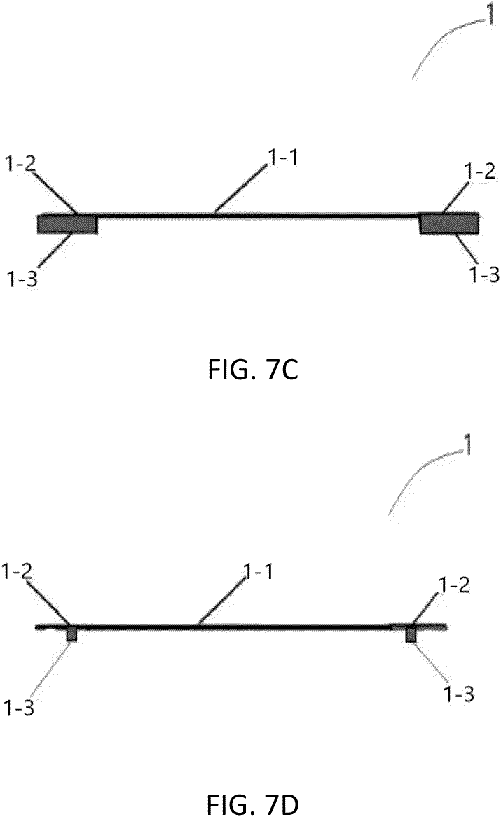

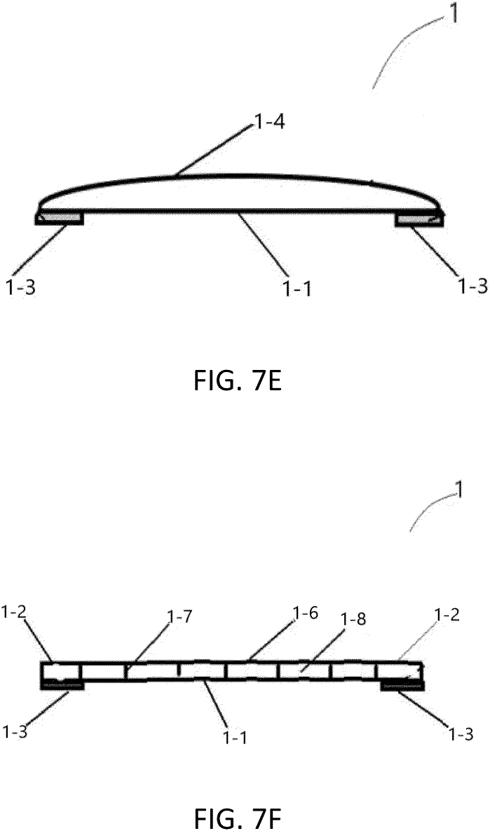

FIGS. 7A to 7F is schematic structure of an outer layer of an IBD according to an example of the present application.

As shown in FIG. 7A, the outer layer 1 can be a transparent film, which can comprise a core zone 1-1 and a marginal zone 1-2, a size of the core zone 1-1 is consistent with the inter layer 2, the marginal zone 1-2 is the outer layer 1 circumferential margin beyond the inter layer 2 part, where, the outer layer 1 can be made with transparent and soft medical polymer materials or medical silicone with a thickness from 0.1 mm to 1.0 mm, preferably from 0.2 mm to 0.5 mm, which can ensure that the outer layer 1 has sufficient resistance to traction toughness without a bloat. In addition, the marginal zone 1-2 has a width from 5 mm to 25 mm, preferably from 10 mm to 15 mm, and the marginal zone 1-2 has a smooth side and an adhesive side for sticking to the wound surrounding healthy skin.

As shown in FIG. 7B, the outer layer 1 can comprise a core zone 1-1, a marginal zone 1-2, and a compartment part 1-3 which can be independently made and then integrated with outer layer 1. An upper edge of the compartment parts 1-3 can be planar and fused with a lower edge of the marginal zone 1-2, an outer edge of the compartment part 1-3 can be a wedge-shaped narrow part, an inner edge of compartment part 1-3 can be a wedge-shaped wide part, in which, a height of the compartment part 1-3 can be slightly higher than the height of the interlayer 2, and a lower edge of the compartment part 1-3 can be coated with adhesive for sticking to healthy skin around the wound.

As shown in FIG. 7C, the outer layer 1 can comprise a core zone 1-1, a marginal zone 1-2, and a compartment part 1-3 which can be independently made and then integrated with outer layer 1. Of course, the compartment part 1-3 can also be integrated with the outer layer 1. An upper edge of the compartment part 1-3 can be fused with a lower edge of the marginal zone 1-2, and a lower edge of the compartment part 1-3 can be coated adhesive for sticking to healthy skin around the wound.

As shown in FIG. 7D, the outer layer 1 can comprise a core zone 1-1, a marginal zone 1-2, and a compartment part 1-3 which can be independently made and then integrated with outer layer 1. The core zone 1-1 dimension can be consistent with the interlayer 2, the marginal zone 1-2 can be beyond a peripheral margin part of the outer layer 1. An upper side of the marginal zone 1-2 can be a smooth surface, and a lower side of the marginal zone 1-2 can be coated with adhesive for sticking to t healthy skin around the wound. In addition, the compartment part 1-3 can be set in a lower side of the marginal zone 1-2 close to the core zone 1-1. A section appearance of the compartment part 1-3 can be rectangular, set in a circumferential margin of the outer layer 1, with a height slightly higher than the interlayer 2, and with a width from 0.5 mm to 5 mm, and with a grooved area in the core zone 1-1 site configured to accommodate the interlayer 2. The compartment part 1-3 could be independently fabricated and then integrated with the outer layer 1, or be made integrate with the outer layer 1.

As shown in FIG. 7E, the outer layer 1 can comprise a core zone 1-1, an air bag 1-4, and a compartment part 1-3. The core zone 1-1 can be consistent with that of the interlayer 2, and the air bag 1-4 can be a superstructure of the outer layer 1 configured to protect the wound when inflated. The compartment part 1-3 can be a lower peripheral structure of the outer layer 1 with a rectangular appearance, with a height slightly higher than that of the interlayer 2, and a width from 5 mm to 25 mm coated with adhesive at a lower side configured to stick healthy skin around the wound, so as to form a groove area corresponding to the core zone 1-1 configured to accommodate the interlayer 2.

As shown in FIG. 7F, the outer layer 1 can comprise a core zone 1-1, a marginal zone 1-2, a compartment part 1-3, a protective film 1-6, a column 1-7, and a chamber 1-8. The core zone 1-1 can be consistent with the interlayer 2. The marginal zone 1-2 can be a peripheral part of the outer layer 1 beyond the interlayer 2, and a lower side of the marginal zone 1-2 can be the compartment part 1-3. The compartment part 1-3 can be set at a peripheral edge of the outer layer 1 with rectangular appearance, and with a height slightly higher than that of the interlayer 2, so that a groove area can be formed corresponding to the core zone 1-1 configured to accommodate the interlayer 2. The compartment part 1-3 can be coated with adhesive under a side for sticking to healthy skin around the wound. The protective film 1-6, the column 1-7 and the chamber 1-8 can be a superstructure of the outer layer 1 configured to protect the wound.

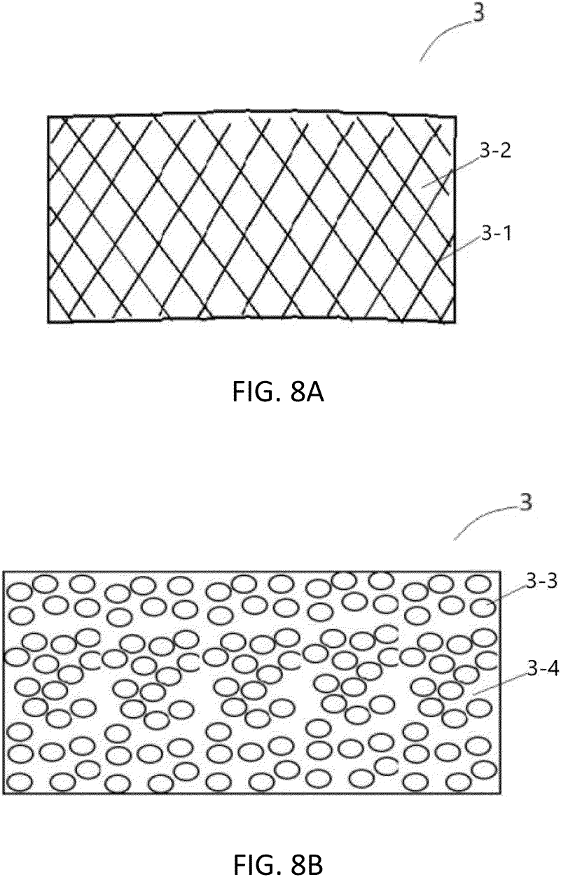

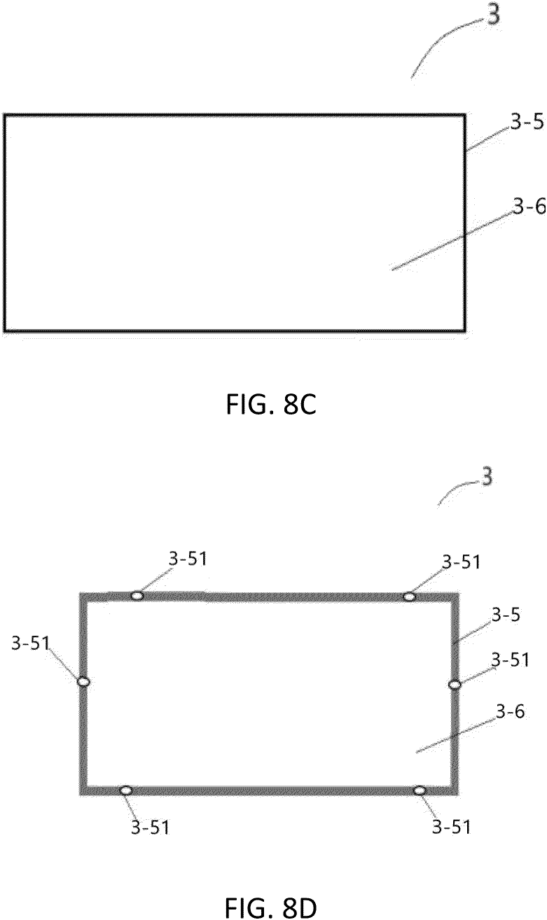

FIGS. 8A to 8D are schematic views illustrating an inner layer structure of an IBD according to the preferred embodiment of the present application.

As shown in FIG. 8A, the inner layer 3 can be made with mesh material and cropped according to a wound shape and size, usually choose not to bond wound and low sensitive medical synthetic material or medical silicone material for directly covering wound. The inner layer 3 can comprise a net 3-1 with a thickness not more than 0.5 mm, and a plurality of meshes 3-2 with a pore size not less than 0.02 mm. As shown in FIG. 8B, the inner layer 3 can be made with sieve like materials and cropped according to the wound shape and size, usually choose not to bond the wound and low sensitive medical synthetic materials or medical silicone materials for directly covering the wound. The inner layer 3 can comprise a sieve frame 3-4 with a thickness not more than 0.5 mm, and a plurality of holes 3-2 with a pore size not less than 0.02 mm.

As shown in FIG. 8C, the inner layer 3 can be set up as a frame structure according to the wound shape and size configured to scaffold normal skin around the wound, and then the interlayer 2 and the outer layer 1 can be placed on the frame structure to avoid the wound from extra foreign body stimulation and protect the wound. The inner layer 3 can include a frame 3-5 with a thickness not more than 0.5 mm, and a plurality of openings 3-6 with a width not more than 5 mm, which usually fabricated from low sensitivity medical synthetic materials or medical silicone materials. In addition, as shown in FIG. 8D, the inner layer 3 can also be set a plurality of jacks 3-51 on the frame 3-5 configured to firmly link with the interlayer 2, and stick healthy skin around the wound with adhesive on a side of the frame 3-5.

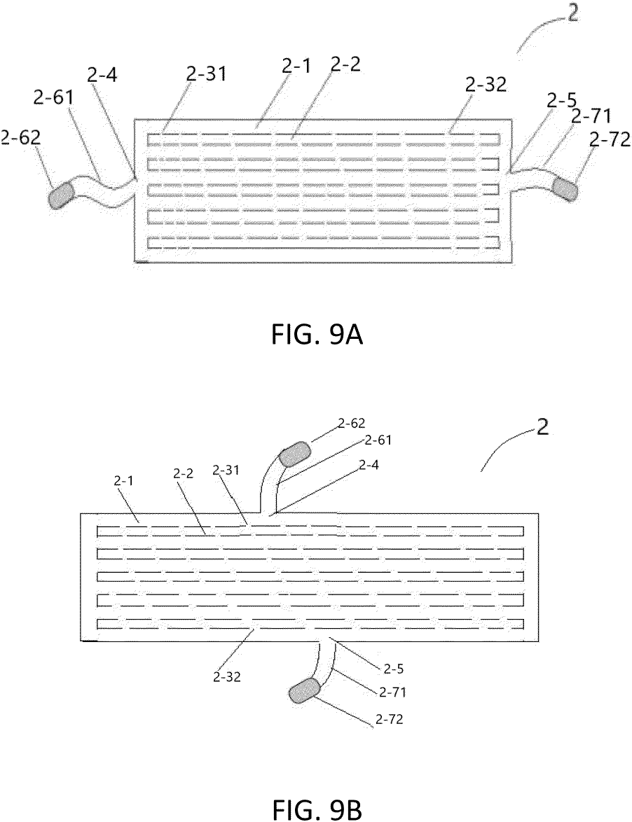

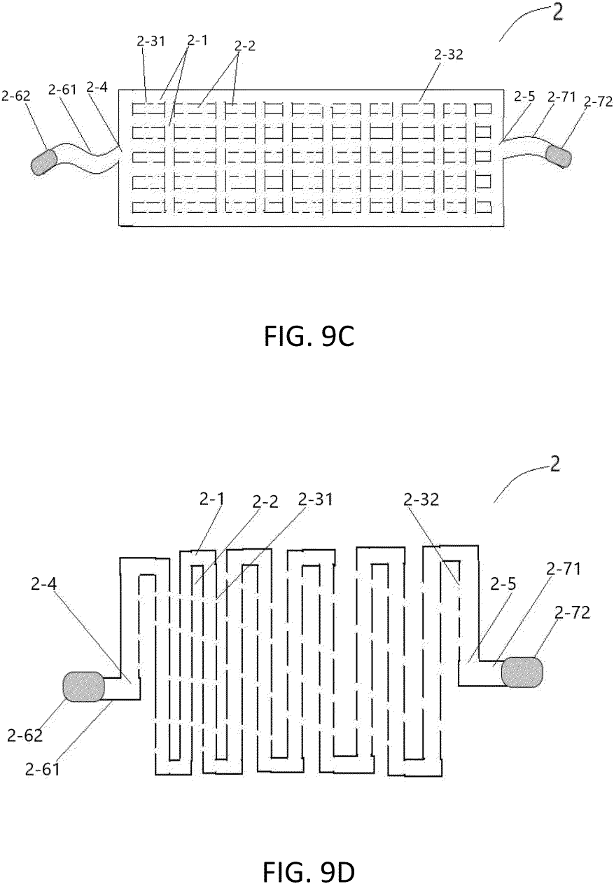

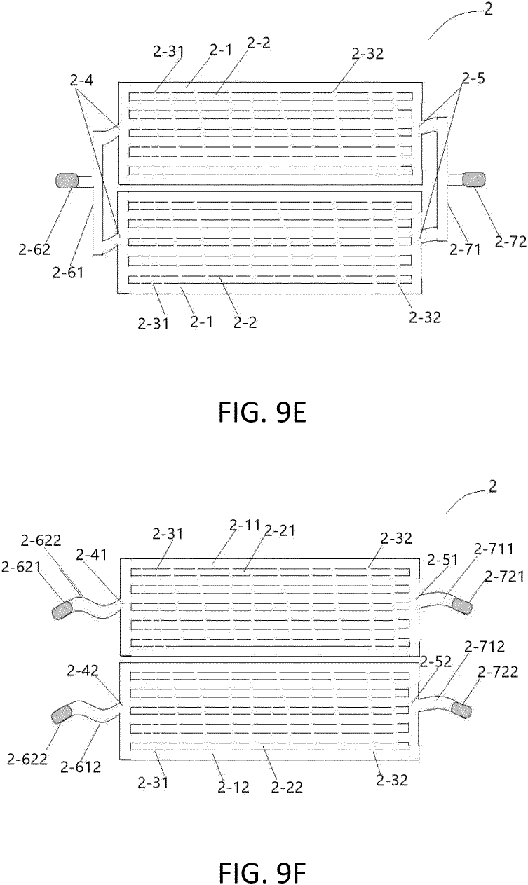

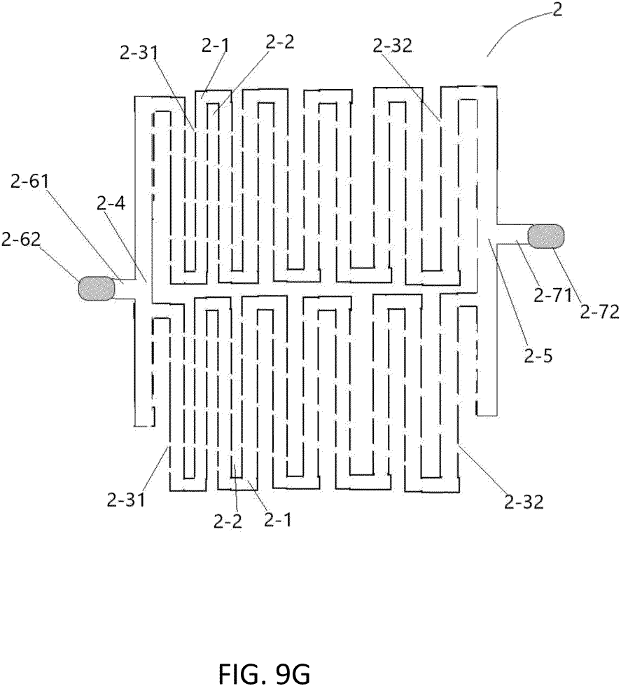

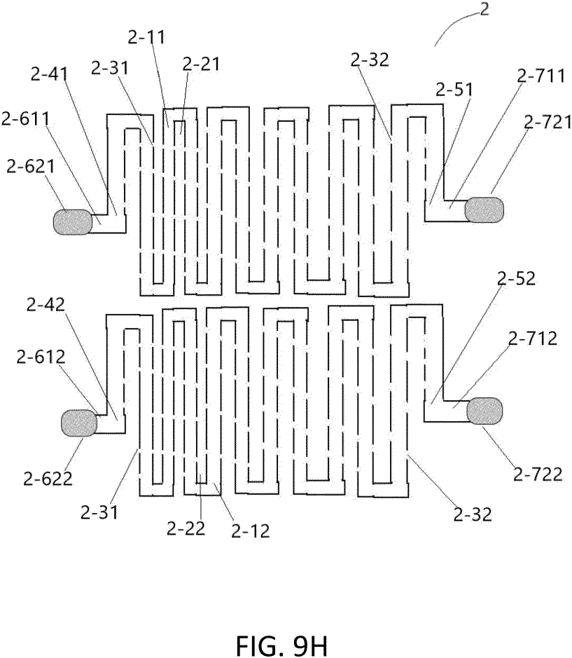

Moreover, with reference to FIGS. 8C, 8D and 9A, the frames 3-5 of the inner layer 3 can directly fuse with the capillary 2-1 or the connecting tube 2-61 and/or 2-71 of the microfluidic system into an integrated structure, which can further simplify the IBD structure and reduce a cost. Similarly, with reference to FIGS. 7B, 7F and 9A, the compartment parts 1-3 can directly fuse with the capillary 2-1 or the connecting tube 2-61 and/or 2-71 of the microfluidic system into an integrated structure, which can further simplify the IBD structure and reduce the cost.

FIGS. 9A to 9D are schematic structures of a microfluidic system as an inter layer main architecture according to an example of the present application.

As shown in FIG. 9A, the microfluidic system can exhibit a rectangular appearance, which can include a first port 2-4, a plurality of capillaries 2-1, a plurality of gaps 2-2, a plurality of proximal second ports 2-31, a plurality of distal second ports 2-32, a terminal port 2-5, a first junction 2-61, a first interface 2-62, a second junction 2-71, and a second interface 2-72, the first port 2-4 can be threaded out of the outer layer 1 through the first junction 2-61, the first interface 2-62 and connected with the filling unit 4, a plurality of capillaries 2-1 can be formed in parallel arranged a capillary 2-1 net, a space between the capillary 2-1 net can be the gaps 2-2, the first port 2-4 can be connected with the capillary 2-1, the proximal second port 2-31 and the distal second port 2-32 being scattered set at tube wall pores of the capillary 2-1, the proximal second port 2-31 can be set at adjacent the first port 2-4 region of the capillary 2-1, the distal second port 2-32 can be set at adjacent terminal port 2-5 region of the capillary 2-1, and the terminal port 2-5 can be threaded through the outer layer 1 to connect with the waste liquid collecting unit 8 via a second connecting tube 2-71 and the second interface 2-72. An artificial amniotic fluid can enter the capillary 2-1 net through the filling unit 4, the first interface 2-62, the first connection tube 2-61, and the first port 2-4, and some artificial amniotic fluid can enter the gap 2-2 through the proximal second port 2-31 or the distal second port 2-32, and penetrate into the inner layer 3 through the gap 2-2, which can wet the wound, and the gap 2-2 internal divided artificial amniotic fluid can reenter the capillary 2-1 through the distal second port 2-32 and can pass through the second connection tube 2-71 and the second interface 2-72 into the waste liquid collection unit 8 to achieve unidirectional flow of artificial amniotic fluid within the microfluidic system.

In addition, the capillary 2-1 can be made with transparent or soft medical polymer materials or medical silica gel, and an outer wall of the capillary 2-1 can be set up with a coating material with a bacteriostatic effect.

In addition, the capillary 2-1 can be with an inner diameter from 0.1 mm to 0.8 mm, preferably from 0.2 mm to 0.5 mm, with a wall thickness from 0.01 mm to 1.0 mm, preferably from 0.05 mm to 0.25 mm, with a spacing from 0.2 mm to 2.0 mm, preferably from 0.5 mm to 1.0 mm. The space between the proximal second port 2-31 and the distal second port 2-32 can be from 0.5 mm to 1.0 mm within the capillary 2-1 net. An aperture can be set from 0.02 mm to 0.1 mm in the proximal second port 2-31, and from 0.1 mm to 0.2 mm in the distal second port 2-32.

Understandably, the microfluidic systems can be either rectangular in appearance or square or circular or irregular in shape, and the capillary 2-1 nets of the microfluidic system can be planar structure in order to adapt to a close to planar structure wound, such as a flat torso site, a hand palm or back, or can be shaped as stereological structure to accommodate non planar structure wounds, such as a head, a face, an extremity. In addition, the microfluidic system with an accessible size or slightly smaller than the inter layer 2 can be used for a narrow and long surgical incision wound, and also be used for large irregular burn scald wounds or chronic wounds.

As shown in FIG. 9B, the microfluidic system can comprise a first port 2-4, a plurality of capillaries 2-1, a plurality of gaps 2-2, a plurality of proximal second ports 2-31, a plurality of distal second ports 2-32, a terminal port 2-5, a first junction 2-61, a first interface 2-62, a second junction 2-71, and a second interface 2-72, the first end port 2-4, the first connection tube 2-61 and the first interface 2-62 can be set at an upper end of the microfluidic system, and the terminal port 2-5, the second connection tube 2-71 and the second interface 2-72 can be set at a lower end of the microfluidic system. In addition, the terminal port 2-5, the second connecting tube 2-71 and the second interface 2-72 can also be set at a left or right end of the microfluidic system.

As shown in FIG. 9C, the capillary 2-1 net of the microfluidic system can further comprise a traffic branch capillary 2-1, which is beneficial to accelerate an entry of artificial amniotic fluid into the capillary 2-1 net by accelerating the filling unit 4, the first interface 2-62, the first connection tube 2-61 and the first port 2-4, thus enhancing a moist wound efficiency.

As shown in FIG. 9D, the capillary 2-1 of the microfluidic system could have only one piece, which can be bent to form the capillary 2-1 net, and the artificial amniotic fluid can be penetrated into the gap 2-2 through the proximal second port 2-31 and the distal second port 2-32 configured to wet the wound, and be pooled into the waste liquid collection unit 8 through the proximal second port 2-31 and the distal second port 2-32.

As shown in FIG. 9E, the microfluidic system can comprise two sets of capillaries 2-1 nets in parallel, which share the first interface 2-62, the first connecting tube 2-61, the second connecting tube 2-71 and the second connecting tube 2-72. Understandably, the microfluidic systems can comprise three or more sets of capillaries 2-1 nets, and each set of capillary 2-1 nets can be identical or different in morphology and structure. In addition, as shown in FIG. 9F, the microfluidic system can comprise two sets of microfluidic branching systems, which can include a first port 2-41 or first port 2-42, a capillary 2-11 or capillary 2-12, a gap 2-21 or gap 2-22, a proximal second port 2-31, a distal second port 2-32, a terminal port 2-51 or terminal port 2-52, a first tube 2-611 or first tube 2-612, a first interface 2-621 or first interface 2-622, a second connecting tube 2-711 or second connecting tube 2-712, and a second connecting tube 2-721 or second connecting tube 2-722, respectively. The two sets of microfluidic branching systems can be a same or different size and shape configured to cover a plurality of irregular wounds.

As shown in FIG. 9G, the microfluidic system can comprise two sets of capillary 2-1 net formed by bending one piece of capillary 2-1, which can share the first interface 2-62, the first connecting tube 2-61, the second connecting tube 2-71, and the second connecting tube 2-72. Understandably, the microfluidic systems can comprise three or more sets of capillary 2-1 nets formed by bending one piece of capillary 2-1 with the same or different morphology and structure. In addition, as shown in FIG. 9H, the microfluidic system can comprise two sets of microfluidic branching systems, which can include a separate capillary 2-1 net formed by bending one piece of capillary 2-1, and each set of microfluidic branching system can include a first port 2-41 or first port 2-42, a capillary 2-11 or capillary 2-12, a gap 2-21 or gap 2-22, a proximal second port 2-31, a distal second port 2-32, a terminal port 2-51 or terminal port 2-52, a first connection tube 2-611 or first connection tube 2-612, a first interface 2-621 or first interface 2-622, a second interface 2-711 or second interface 2-712, and a second interface 2-721 or second interface 2-722. The two sets of microfluidic branching systems can be a same or different size configured to cover a plurality of irregular wounds.

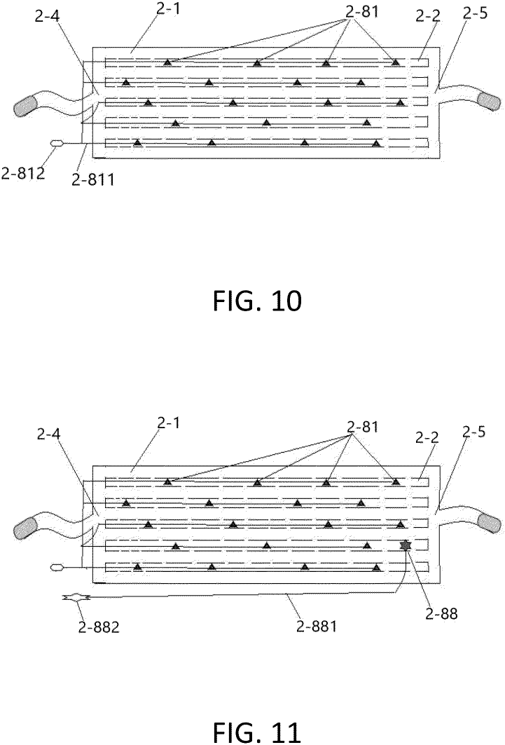

FIG. 10 is a schematic structure of an interlayer with a heating unit of an IBD according to an example of the present application. As shown in FIGS. 1, 9A, and 10, the interlayer can comprise a heating unit 6, which can include a plurality of heating components 2-81, a first cable 2-811 and a first plug 2-812, the heating component 2-81 can be distributed within a capillary 2-11 net gap 2-2 configured to heat artificial amniotic fluid within gap 2-2. The heating component 2-81 can be electrically connected with a control unit 9 and a power supply 7 through the first cable 2-811 and the first plug 2-812. The control unit 9 can real time process the artificial amniotic fluid temperature data within the gap 2-2 acquired by a monitoring unit 5, and command the heating components 2-81 to work so that the artificial amniotic fluid within the gap 2-2 maintains a constant temperature condition from 36.5° C. to 37.5° C.

FIG. 11 is a schematic view illustrating a structure of an interlayer with a plurality of lamp tubes of an IBD according to an embodiment of the present application. As shown in FIGS. 1, 9A, 10 and 11, a gap 2-2 of an interlayer 2 can comprise a heating unit 6 and a plurality of lamp tubes 2-88, the lamp tubes 2-88 can be connected with a control unit 9 and a power supply 7 pluggable through a ninth cable 2-881 and a ninth plug 2-882. The lamp tubes 2-88 can be scattered in the gap 2-2 between a capillary 2-1 nets, and the lamp tubes 2-88 can emit a variety of energy lights such as infrared, far infrared, and ultraviolet light, which can be configured to provide physical therapy, anti-infective treatment for wounds. Because the lamp tubes 2-88 and its production, such as light or ozone, are confined to a tiny space, and less than 1 mm away from the wound, so that the tubes 2-88 require only a small amount of energy consumption to exert a safe and effective treatment effect.

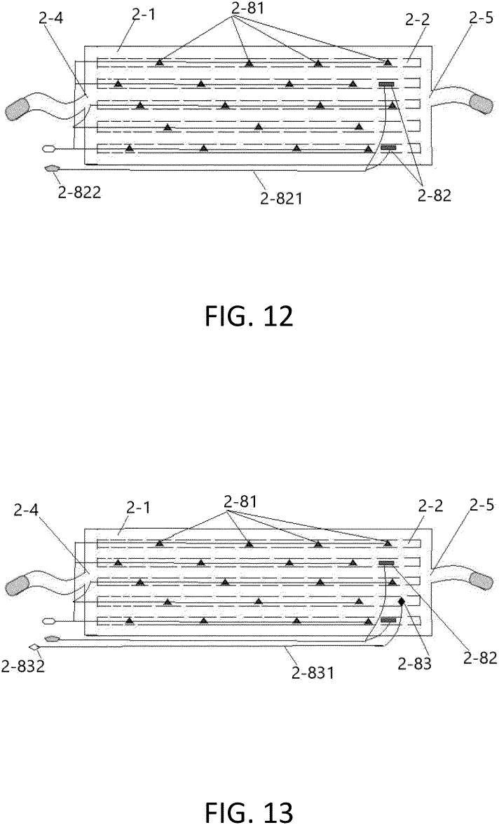

FIGS. 12 to 18 is a schematic view illustrating an interlayer with a plurality of sensors of an IBD according to an embodiment of the present application. As shown in FIGS. 1, 9A, 10 and 12, a gap 2-2 of an interlayer 2 can comprise a heating unit 6 and a plurality of temperature sensors 2-82 configured to collect artificial amniotic fluid temperature data within the gap 2-2 and connect to a control unit 9 and a power supply 7 through a second cable 2-821 and a second plug 2-822. As shown in FIGS. 1, 9A, 10, 12 and 13, a gap 2-2 of an interlayer 2 can comprise a heating unit 6, a plurality of temperature sensors 2-82, and a pH sensor 2-83, the pH sensor 2-83 can be configured to collect artificial amniotic fluid pH data within the gap 2-2 and connected to a control unit 9 and a power supply 7 through a third cable 2-831 and a third plug 2-832.

As shown in FIGS. 1, 9A, and 12 to 14, a gap 2-2 of an interlayer 2 can comprise a heating unit 6, a plurality of temperature sensors 2-82, a pH sensor 2-83, and a humidity sensor 2-84, the humidity sensor 2-84 can be configured to collect humidity data within the gap 2-2 and connected to a control unit 9 and power supply 7 through a fourth cable 2-841 and a fourth plug 2-842. As shown in FIGS. 1, 9A, 10 and 12 to 15, a gap 2-2 of an interlayer 2 can comprise a heating unit 6, a plurality of temperature sensors 2-82, a pH sensor 2-83, a humidity sensor 2-84, and a plurality of microbial sensors 2-85, the microbial sensors 2-85 can be configured to collect microbial metabolic ATP data within the gap 2-2 and connected to a control unit 9 and power supply 7 through a fifth cable 2-851 and a fifth plug 2-85-2.

As shown in FIGS. 1, 9A, 10, and 12 to 16, a gap 2-2 of an interlayer 2 can comprise a heating unit 6, a plurality of temperature sensors 2-82, a pH sensor 2-83, a humidity sensor 2-84, and a plurality of oxygen saturation sensors 2-86, the oxygen saturation sensors 2-86 can be configured to collect wound tissue intravascular oxygen saturation data and electrically connected to a control unit 9 and a power supply 7 through a sixth cable 2-861 and a sixth plug 2-862.

As shown in FIGS. 1, 9A, 10 and 12 to 17, a gap 2-2 of an interlayer 2 can comprise a heating unit 6, a plurality of temperature sensors 2-82, a pH sensor 2-83, a humidity sensor 2-84, a plurality of oxygen saturation sensors 2-86, and a pressure sensor 2-87, the pressure sensor 2-87 can be configured to collect pressure data on a surface of the wound and electrically connected to a control unit 9 and a power supply 7 through a seventh cable 2-871 and a seventh plug 2-872.

As shown in FIGS. 1, 9A, 10 and 18, a gap 2-2 of an interlayer 2 can comprise a heating unit 6, and a set of integrated sensors 2-9. The integrated sensors 2-9 can comprise a combination of at least one or more temperature sensor 2-82, pH sensor 2-83, humidity sensor 2-84, microbial sensor 2-85, oxygen saturation sensor 2-86 and pressure sensor 2-87, which can be configured to collect temperature or / and pH or / and humidity or / and oxygen saturation or / and pressure data, and can be connected to a control unit 9 and power supply 7 through a eighth cable 2-91 and an eighth plug 2-92.

It is needed to note that the sensor species, quantity, and layout in the gap 2-2 of the interlayer of an IBD according to an example of the present application can be set on a demand. The temperature sensor 2-82, pH sensor 2-83, humidity sensor 2-84, microbial sensor 2-85, oxygen saturation sensor 2-86, pressure sensor 2-87 sensor, and integrated sensor 2-9 and their cables or plugs can be recyclable used, so as to avoid unnecessary waste. In addition, the data collected by temperature sensor 2-82, pH sensor 2-83, humidity sensor 2-84, microbial sensor 2-85, oxygen saturation sensor 2-86, pressure sensor 2-87 sensor, and integrated sensor 2-9 can be transmitted to a cloud server or a smart terminal of a user or medical care specialist through the control unit 9 for intelligent decision-making of the IBDs.

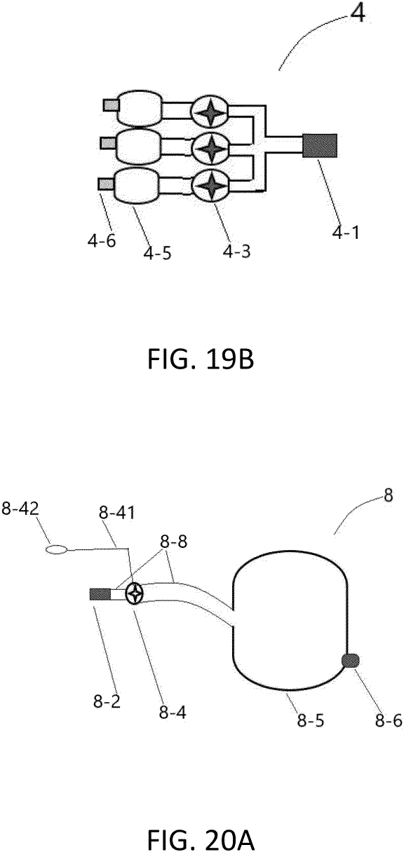

FIGS. 19A and 19B are schematic views illustrating a structure of a filling unit of an IBD according to an embodiment of the present application.

As shown in FIGS. 4 and 19A, a filling unit 4 can comprise a third interface 4-1, a first smart pump 4-3, a plurality of first smart valves 4-4, a plurality of reservoir vessels 4-5, and a plurality of first injection pots 4-6. The third interface 4-1 can be pluggable connected with the first interface 2-62 of a microfluidic system, and a syringe can inject artificial amniotic fluid or additives into the reservoir 4-5 through the first injection pots 4-6, then the artificial amniotic fluid or additives can enter the microfluidic system through the first smart valves 4-4, the first smart pump 4-3, the third interface 4-1 and the first interface 2-62. It needs to be illustrated that there are three first smart valves 4-4 and three reservoir vesicles 4-5 being set up in the filling unit 4 as shown in FIG. 19A, actually, one, two or more first smart valves 4-4 and reservoir vesicles 4-5 can be set up according to a demand. In addition, when the filling unit 4 receives a control unit 9 or an artificial control start instruction, in which at least one of the first smart valves 4-4 and the first smart pump 4-3 can be started, so that artificial amniotic fluid or additives inside the reservoir 4-5 can be injected into the microfluidic system; when the filling unit 4 receives the control unit 9 or artificial control stop instruction, the first smart valve 4-4 and the first smart pump 4-3 can stop at the same time. In addition, the reservoir 4-5 can store artificial amniotic fluid, buffers, anesthetics, growth factors or stem cells, lytic enzymes, anti-infective agents, etc., configured to provide a moist, slightly acidic environment for the wound, or to reduce wound pain, accelerate wound healing, and treat wound infection. In addition, a volume measurement device can be set in the reservoirs 4-5, which can emit a prompt to instill artificial amniotic fluid, buffer or other additives into the reservoir 4-5 in a timely manner as the fluid within the reservoir 4-5 approaches a minimum volume.

As shown in FIGS. 4 and 19B, a filling unit 4 can comprise a third interface 4-1, a plurality of first smart pumps 4-3, a plurality of reservoir vessels 4-5, and a plurality of first injection pots 4-6, the third interface 4-1 can be pluggable connect with the first interface 2-62 of the microfluidic system, a syringe can be used to inject artificial amniotic fluid or additives into the reservoir 4-5 through the first injection pots 4-6, the reservoir fluid or additives can be transferred through the first smart pump 4-3, the third interface 4-1 and the first interface 2-62 into the microfluidic system. It should be stated that three first smart pumps 4-3 and three reservoir sacs 4-5 are set up for the filling unit 4 in FIG. 19B, actually, one, two or more first smart pumps 4-3 and reservoirs 4-5 can be set up in a pluggable manner as needed. In addition, when the filling unit 4 receives a control unit 9 or artificial control start instruction, at least one first smart pump 4-3 is started, so that the artificial amniotic fluid or additives inside the reservoir 4-5 can be injected into the microfluidic system, when the filling unit 4 receives the control unit 9 or artificial control stop instruction, the first smart pump 4-3 can stop working.

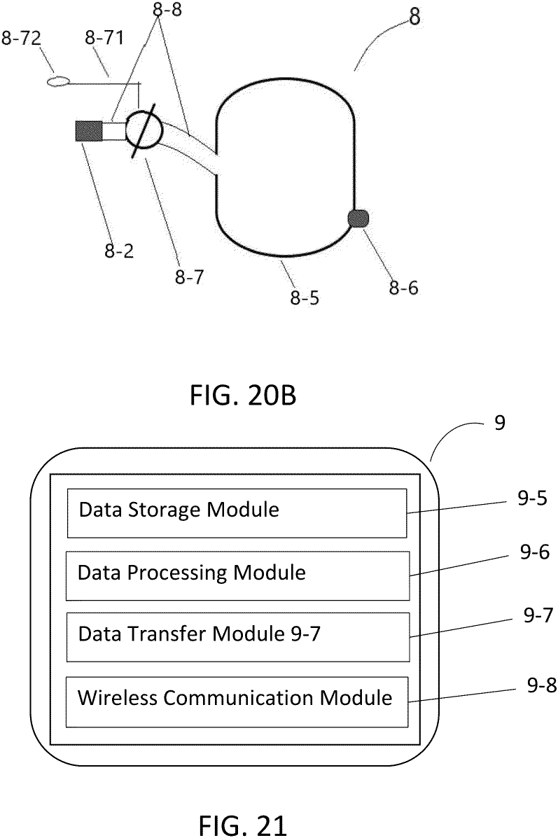

FIGS. 20A and 20B are schematic views illustrating a structure of a waste liquid collection unit of an IBD according to an embodiment of the present application.

As shown in FIGS. 1, 4, 9A, and 20A, a waste liquid collection unit 8 can comprise a fourth connection tube 8-8, a fourth interface 8-2, a second smart pump 8-4, a tenth cable 8-41, a tenth plug 8-42, a waste sac 8-5,and a second injection pot 8-6, the fourth connection tube 8-8 can connect the fourth interface 8-2, the second smart pump 8-4, the second smart pump 8-4 and the waste sac 8-5, the fourth interface 8-2 can pluggable connect with the second interface 2-72 of the microfluidic system, the second smart pump 8-4 can pluggable connect with the waste sac 8-5, and the second smart pump 8-4 can pluggable connect with a control unit 9 and a power supply 7 through the tenth cable 8-41 and the tenth plug 8-42. When the second intelligent pump 8-4 receives the control unit 9 or the artificial control start instruction, artificial amniotic fluid or additives and tissue metabolic components in a capillary 2-1 net, a gap 2-2, an inner layer 3 and the wound can enter the waste sac 8-5 and be stored temporarily, a medical experts can use a syringe to draw liquid inside the waste sac 8-5 through the second injection pot 8-6 for analysis. In addition, the waste sac 8-5 can be set up with a volumetric measuring device that emits a prompt for the timely aspiration of waste or replacement of the waste sac 8-5 when the liquid within the waste sac 8-5 is near the maximum capacity.

As shown in FIGS. 1, 4, 9A and 20B, a waste liquid collection unit 8 can comprise a fourth connection tube 8-8, a fourth interface 8-2, a second smart valve 8-7, a eleventh cable 8-71, a eleventh plug 8-72, a waste sac 8-5, and a second injection pot 8-6, the fourth connection tube 8-8 can connect the fourth interface 8-2, the second smart valve 8-7, the second smart valve 8-7 and the waste sac 8-5, the fourth interface 8-2 can pluggable connect with a second interface 2-72 of the microfluidic system, the second smart valve 8-7 can pluggable connect with the waste sac 8-5, the second smart valve 8-7 can connect with a control unit 9, and the eleventh plug 8-72 can pluggable connect with the control unit 9. When the second smart valve 8-7 receives the control unit 9 or an artificial control start instruction, artificial amniotic fluid or additives and tissue metabolic components inside a capillary 2-1 net, a gap 2-2, an inner layer3 as well as the wound bed can enter the waste sac 8-5 and store temporarily, when the second smart valve 8-7 receives the control unit 9 or the artificial control stop instruction, the temporary storage fluid inside the waste sac 8-5 cannot reflux into the microfluidic system.

FIG. 21 shows a schematic illustration of a control unit of an IBD of an embodiment of the present application.

As shown in FIGS. 1, 4, 5 and 21, a control unit 9 can comprise a data storage module 9-5, a data processing module 9-6, a data transfer module 9-7 and a wireless communication module 9-8. The control unit 9 can pluggable connect with a filling unit 4, a monitoring unit 5, a heating unit 6, a power supply 7 and a waste liquid collection unit 8. The raw data collected in the monitoring unit 5 can transfer by the data transfer module 9-7 and store in the data storage module 9-5, then an intelligent decision instruction formed by the data processing module 9-6 can be send by the data transfer module 9-7 to the filling unit 4, the heating unit 6 and the waste liquid collection unit 8 configured to intelligent operate the filling unit 4, the heating unit 6 and the waste liquid collection unit 8, at same time, the raw data collected by the monitoring unit 5 and the intelligent decision instruction data formed by the data processing module 9-6 can be uploaded to a cloud server or a medical expert intelligence terminal via the wireless communication module 9-8 configured to download or provide a base for medical experts to implement artificial intervention.

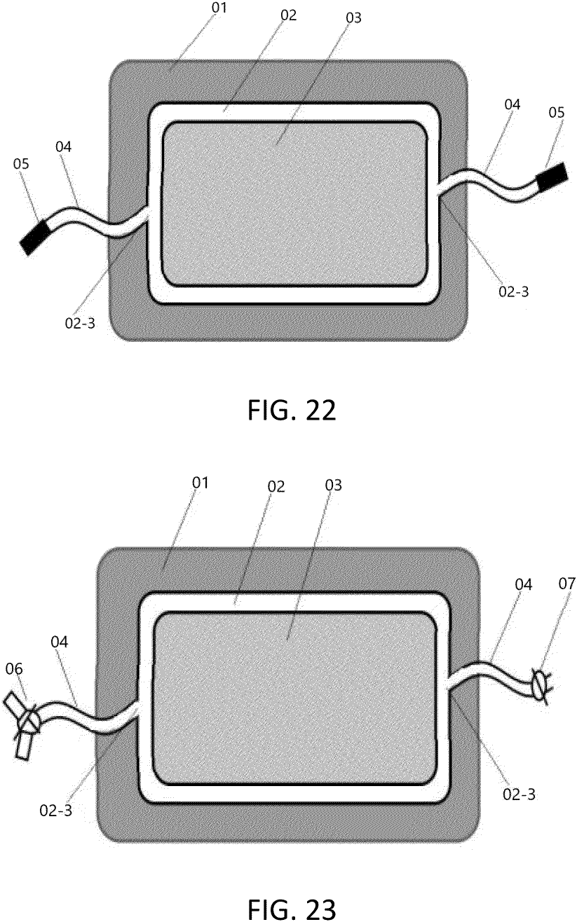

Referring to FIG. 22, a fabrication process 100 of the IBD according to the preferred embodiment of the present invention comprises steps as follow.

S110. Plan an IBD including a model, a category, and a plurality of functional units as required.

Referring to FIGS. 1 to 3, IBD functional units can include, but are not limited to, an outer layer 1, an interlayer 2, an inner layer 3, a filling unit 4, a monitoring unit 5, a heating unit 6, a power supply 7, a waste liquid collection unit 8, a control unit 9, and a patch membrane 10. With reference to planning, components required for IBD functional units can be designed.

S120. Fabricate an outer layer.

Referring to FIGS. 7A to 7F, the outer layer 1 can be fabricated and divided into a core zone 11, a marginal zone 1-2, a compartment part 1-3, and/or an air bag 1-4, and/or a film 1-6, and/or a column 1-7 according to step S110. Then the core zone 11, the marginal zone 1-2, the compartment part 1-3, and/or the air bag 1-4, and/or the film 1-6, and/or the column 1-7 can be assembled as the outer layer 1, and the marginal zone 1-2 or compartment part 1-3 coated adhesive. The outer layer 1 can be made of transparent and soft medical polymer material or medical silicone, or other materials.

S130. Fabricate an interlayer of an IBD which comprises a plurality of steps as follow.

S131. Fabricate a microfluidic system. Referring to FIGS. 9A to 9H, the microfluidic system with corresponding sizes and shapes can be fabricated according to the interlayer 2 model designed in step S110, which can include but not limited to a first port 2-4, a capillary 2-1, a gap 2-2, a proximal second port 2-31, a distal second port 2-32, a terminal port 2-5, a first connecting tube 2-61, a first connecting tube 2-62, a second connecting tube 2-71, and a second connecting tube 2-72. A manufacturing processes can include, but are not limited to, injection molding, welding, 3D printing.

In addition, the capillary 2-1 can be made with transparent and soft medical polymer materials or medical silica gel, and an outer wall of the capillary 2-1 can be coated with a bacteriostatic coating.

In addition, the microfluidic system can include, but is not limited to, two or more sets of capillaries 2-1 nets, which can share the first connection tube 2-61, the first interface 2-62, the second connection tube 2-71 and the second interface 2-72, or set up the first connection tube 2-61, the first interface 2-62, the second connection tube 2-71 and the second interface 2-72 independently. Further, the microfluidic system can include, but are not limited to, two or more sets of microfluidic branching systems.

S133. Make a heating unit. Referring to FIGS. 1, 9A, and 10, the heating unit 6 of the interlayer 2 can include, but is not limited to, a plurality of heating components 2-81, a first cable 2-811, and a first plug 2-812. The heated components 2-81 can be scattered throughout a gap 2-2 of the capillary 2-1 net, and bonded to an outer wall of the capillary 2-1.

S135. Make a lamp tube. Referring to FIGS. 1, 9A and 11, the interlayer 2 can comprise a plurality of lamp tubes 2-88, a ninth cable 2-881, and a ninth plug 2-882. The lamp tubes 2-88 can be scattered in a gap 2-2 between a capillary 2-1 nets, which can emit including but not limited to infrared, far infrared, and/or ultraviolet light.

S137. Make a monitoring unit. Referring to FIGS. 1, 9A and 12 to 18, the interlayer 2 can set up at least one temperature sensor 2-82, a second cable 2-821, a second plug 2-822, at least one pH sensor 2-83, a third cable 2-831, a third plug 2-832, at least one humidity sensor 2-84, a fourth cable 2-841, a fourth plug 2-842, at least one microbial sensor 2-85, a fifth cable 2-851, a fifth plug 2-852, at least one oxygen saturation sensor 2-86, a sixth cable 2-861, a sixth plug 2-862, at least one pressure sensor 2-87, a seventh cable 2-871, and a seventh plug 2-872. In addition, the interlayer 2 can set up an integrated sensors 2-9, which can electrically connect with a control unit 9 and a power supply 7 through an eighth cable 2-91 and an eighth plug 2-92.

It should be stated that, according to a design objective, the temperature sensor 2-82, pH sensor 2-83, humidity sensor 2-84, microbial sensor 2-85, oxygen saturation sensor 2-86, pressure sensor 2-87 sensor, integrated sensor 2-9 and their cables or plugs can adopt recyclable and reuse engineering standards to avoid unnecessary waste.

S139. Combine or assemble the microfluidic system with the heating unit and/or lamp tubes and/or monitoring unit. Referring to FIGS. 1, 4, 9A and 11 to 18, the microfluidic system can be assembled with the heating components 2-81, temperature sensor 2-82, pH sensor 2-83, humidity sensor 2-84, microbial sensor 2-85, oxygen saturation sensor 2-86, pressure sensor 2-87 sensor, lamp tubes 2-88, integrated sensor 2-9 and their cables or plugs as an integrated interlayer 2.

In addition, referring to FIGS. 1, 4, 9B to 9H, and 11 to 18, the integrated interlayer 2 can be assembled with more than one microfluidic branching systems or a plurality of capillary 2-1 nets in parallel or in series, which can set up a heating components 2-81, temperature sensor 2-82, pH sensor 2-83, humidity sensor 2-84, microbial sensor 2-85, oxygen saturation sensor 2-86, pressure sensor 2-87 sensor, integrated sensor 2-9 and their cables or plugs, so as to bond and fix to construct as an all-in-one interlayer 2.

S140. Fabricate an inner layer of the IBD.

Referring to FIGS. 8A and 8B, the inner layer 3 can comprise a mesh 3-1 with a plurality of pores 3-2, which can be made with mesh material or sieve material depending on a design large sheet material.

Referring to FIGS. 8C and 8D, the inner layer 3 can include, but not limited to, a frame 3-5, a plurality of openings 3-6, and a plurality of jacks 3-51, and the frame 3-5 can be coated with adhesive on a side. In addition, referring to FIGS. 8C, 8D, 9A and 7B to 7F, the frame 3-5 can directly fuse with the microfluidic system of an interlayer 2 into an all-in-one structure, or replace the compartment part 1-3 of an outer layer 1 so as to simplify the IBD structure, thereby reducing fabrication cost.

S150. Fabricate a filling unit of the IBD.

Referring to FIGS. 19A and 19B, the filling unit 4 that is designed according to step S110 can include, but are not limited to, a third interface 4-1, a first smart pump 4-3, a first smart valve 4-4, a plurality of reservoirs 4-5. The third interface 4-1, the first smart pump 4-3, the first smart valve 4-4, the reservoirs 4-5 and the first injection pot 4-6 can be modular parts which can be combined or disassembled on demand. In addition, a universal connection or extension, multi-pass tubes can be made for pluggable splice combinations of modular parts.

S160. Fabricate a control unit of the IBD.

Referring to FIGS. 1, 4, 5 and 21, the control unit 9 can includes but is not limited to a data storage module 9-5, a data processing module 9-6, a data transfer module 9-7, and a wireless communication module 9-8, which can be set a pluggable interface configured to connect with a filling unit 4, a monitoring unit 5, a heating unit 6, a power supply 7, and a waste liquid collection unit 8. In addition, the control unit 9 need an operation code, an application and an algorithm configured to run.

S170. Fabricate a waste liquid collection unit of the IBD.

Referring to FIGS. 4, 9A, 20A and 20B, the waste liquid collection unit 8 designed according to the step S110 can include, but is not limited to, a fourth interface 8-2, a second smart pump 8-4, a second smart valve 8-7, a waste sac 8-5 and a second injection pot 8-6, which can be modular parts with a pluggable interface.

S180. Assemble the IBD which includes a plurality of steps as follow.

S182, combining a core module of the IBD. Referring to FIGS. 1 to 4, an interlayer 2 and an inner layer 3 can be set up on a basis of an outer layer 1, then the outer layer 1, interlayer 2 and inner layer 3 can be bonded or chimeric fixed in turn, if the inner layer 3 adopts a frame 3-1 structure, the inner layer 3 can be pre bonded or chimeric fixed with the interlayer 2 as an integrated structure, and then bonded with the outer layer 1.

S184. Thread a connection tube and cable through the outer layer. As shown in FIG. 4, a first hole can be made at one end of the outer layer 1, and part of the connection tubes and cables thread through the first hole, then a second hole can be made at another end of the outer layer 1, and the other connection tubes and cables thread through the second hole.

S186. Type a peripheral module together with the IBD. As shown in FIG. 5, a charging unit 4 can electrically connect with a control unit 9 and a power supply 7, a heating unit 6 can electrically connect with the control unit 9 and power supply 7, and a waste liquid collection unit 8 can electrically connect with the control unit 9 and power supply 7, and then the control unit 9 can electrically connect with the power supply 7.

S188. Assemble the complete IBD. Referring to FIGS. 1, 3, 4 and 5, a monitoring unit can electrically connect with the control unit 9 and the power supply 7, and the microfluidic system can electrically connect with the filling unit 4 and the waste liquid collection unit 8, then part of the outer layer 1 beyond the interlayer 2 can be coated with adhesive, and then past a patch membrane 10.

S190. Encapsulate and sterilize the IBD.

According to a preferred embodiment of the present application, a method 200 of an IBD can comprise the following steps.

S210. Select a model of the IBD. Based on a patient wound type and size, the IBD can be selected, or personalized. It is important to illustrate that the IBD model can be set up as series based on a size of an outer layer, an interlayer, an inner layer, material, component or functional unit combination as well as a difference in configuration of outer shape to meet a need of different sizes of different parts of human body.

S220. Prepare a wound.

S230. Cover the wound with the IBD. A specific step can include, but is not limited to:
(1) covering the wound prepared at step S220 with the inner layer,
(2) connecting a filling unit, a heating unit, a monitoring unit, a control unit, a waste liquid collection unit, and a power supply,
(3) configuring artificial amniotic fluid and additives and injecting into a reservoir of the filling unit,
(4) setting up an intelligent control program or artificial intervention program,
(5) initiating, real-time collecting monitoring data to provide a warm, moist, slightly acidic, anaerobic Sterile, and stressless microenvironment, and if necessary, adjusting formulation of the artificial amniotic fluid or additive,
(6) the patient or medical specialist directly observing the wound through a transparent IBD without changing the dressing, being able to aspirate the waste sac or replacing it in a timely manner. In addition, if the wound prepared at the step S220 could be bloody, a bandage can be appropriately pressurized for a short time, and the monitoring unit can detect the wound pressure. In addition, the filling unit can be usually placed at an upper or higher end of a human wound, and the waste liquid collecting unit can be placed at a lower or lower end of the human wound, which can be beneficial to a gravity of liquid inside the microfluidic system and reduce energy consumption of the filling unit and the waste liquid collecting unit. It is understandable that because the IBD can be light and thin with slight movement restriction to a patient, the patient can wear clothes normally, which can be beneficial for wound preservation and reduce energy consumption of the heating unit.

S240. Remove the IBD when wound healed.

According to another preferred embodiment of the present application, a method 300 of an IBD can comprise the following steps.

S310. Plan an IBD. A specific step can include, but is not limited to: confirming the IBD model, functional unit configuration based on a patient wound type, shape and size.

S320. Fabricate an outer layer and an inner layer of the IBD bedside. A specific step can include, but is not limited to: (1) cropping a large sheet of sterile outer material as the outer layer with a dimension from 5 mm to 20 mm beyond a patient wound edge, (2) cropping the inner layer with a size equal to or slightly larger than the patient wound size, (3) contouring the outer layer and inner layer.

S330. Febricate an interlayer of the IBD. A specific step can include, but is not limited to: (1) selecting one or more sterile packaging of suitable size modular planar or stereo structured microfluidic systems, monitoring unit assembly, heating unit assembly, lamp tubes, monitoring unit assembly, and heating unit assembly, (2) setting the monitoring unit assembly, heating unit assembly, lamp tubes, monitoring unit assembly and heating unit assembly within the microfluidic system adhesive or chimeric combination as the interlayer.

S340. Prepare a wound.

S350. Cover the wound with the IBD, which can include, but is not limited to:
(1) covering the wound prepared at step S340 with the inner layer made at step S320, the interlayer made at step S330, and the outer layer made at step S320,
(2) making holes at both ends of the outer layer, and
(3) a plurality of connecting tubes and cables threading through the holes to connect a microfluidic system with the filling unit, the waste liquid collection unit, the power supply, the monitoring unit, the heating unit, and the control unit. Additionally, part of the outer layer partially gelatinized beyond the inner layer can be pasted with healthy skin around the wound.

S360. Enable the IBD. A specific step can include, but is not limited to:
(1) configurating artificial amniotic fluid and/or additives,
(2) injecting artificial amniotic fluid and/or additives into a reservoir,
(3) setting up intelligent control programs or artificial intervention programs, and
(4) start up with real-time acquisition of monitoring data to provide a warm, moist, slightly acidic, anaerobic, sterile, and pressure free microenvironment to the wound.

S370. Remove the IBD when the wound healed.

Referring to FIGS. 22 to 35, according to a preferred embodiment of the present application, a simulated amniotic cavity biomimetic dressing (SACBD) can comprise a dressing body, a plurality of fittings and a plurality of accessories. The dressing body can comprise a fixation membrane 01, a frame 02, and a cover film 03. The fittings can comprise a tube line 04, an injection pot 05, a three-way 06, and a valve 07. The accessories can comprise a protective membrane 08, a display 09, a syringe 30, an exhaust needle 40, an infusion bag 50, and a urine collection bag 60.

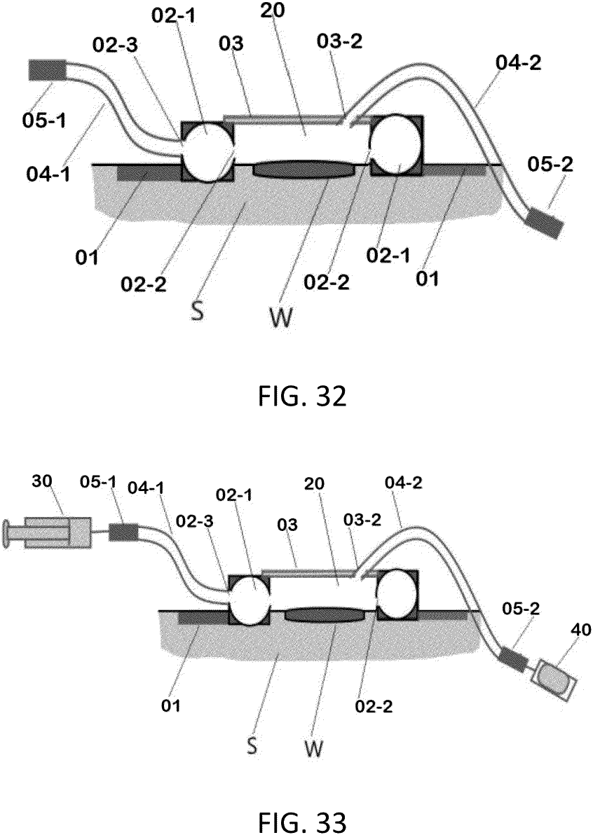

As shown in FIGS. 22 and 32, according to the preferred embodiment of the present application a SACBD can comprise a fixation membrane 01, a frame 02, a cover film 03, a tube line 04, and an injection pot 05. The fixation membrane 01 can encircle the frame 02 and be fixed with an outer edge of the lower end of the frame 02 so as to form an all-in-one structure. The fixation membrane 01 can be smooth frontally and coated with adhesive on a back, with a width no less than 10 mm, preferably from 15 mm to 25 mm, which can be typically made of transparent plastic. The frame 02 can connect to the fixation membrane 01 with an outer edge and the cover film 03 with an upper side edge respectively. The frame 02 can include a plurality of holes 02-3 configured to connect with the tube line 04. The cover film 03 can fix and connect with an upper lateral edge of the frame 02, so that the cover film 03 can firm an all-in-one structure with the frame o2 configured to cover a wound. The cover film 03 can be usually made of transparent plastic with a thickness no more than 2 mm, preferably from 0.5 mm to 1.0 mm. One end of the tube line 04 can connect with the frame 02 through the hole 02-3, and another end of the tube line 04 can connect to the injection pot 05. The tube line 04 can be usually made of transparent plastic with an internal diameter no more than 2 mm, preferably from 0.5 mm to 1.0 mm.

When the SACBD covers a wound, the frame 02 can be set on health skin around the wound, the cover film 03 can keep a space with the wound to prevent compression of the wound bed, and a back adhesive of the fixation membrane 01 can strongly bond with the health skin around the wound, so that an integrated structure composed of the cover film 03 and the frame 02 can form a closed chamber 20 above the wound bed. When artificial amniotic fluid is injected through the injection pot 05, the artificial amniotic fluid can enter into the closed chamber 20 through the tube line 04 and the holes 02-3, thus, a simulated amniotic cavity environment can be created locally at the wound. Of course, the artificial amniotic fluid in the closed chamber 20 can be withdrawn through the injection pot 05, the tube line 04, and the holes 02-3.

In addition, as shown in FIGS. 23 and 32, according to the preferred embodiment of the present application a SACBD can comprise a three-way 06 and a valve 07, so that artificial amniotic fluid and/or additives can be injected into a closed chamber 20 through an injection pot 05, a tube line 04, the three-way 06, the valve 07 and holes 02-3. Of course, the artificial amniotic fluid and/or additives in the closed chamber 20 can be withdrawn through the holes 02-3, the injection pot 05, a tube line 04, the three-way 06, and the valve 07.

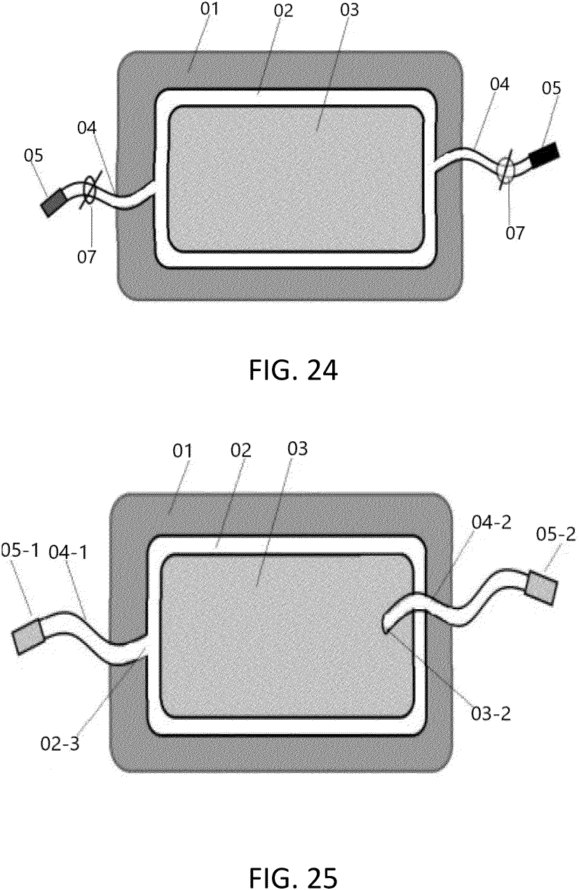

In addition, as shown in FIG. 24, a valve 07 of a SACBD can be set in a middle of a tube line 04 configured to close or open the tube line 04 according to an example of the present application.

It should be noted that tube lines 04 and injection pots 05 of a SACBD as shown in FIGS. 22 to 24 have not label an injection end and/or drainage end of artificial amniotic fluid before use, however, once the SACBD covers a wound, the tube lines 04 and the injection pot 05 of the SACBD should mark the injection end and/or drainage end to ensure that the injection end can be injected with fresh artificial amniotic fluid and/or additives, and the drainage end drained waste liquid, thereby preventing contaminating the wound.

As shown in FIGS. 25 and 32, according to an example of the present application a SACBD can comprise a fixation membrane 01, a frame 02, a plurality of side holes 02-3, a cover film 03, a top hole 03-2, a left tube line 04-1, a right tube line 04-2, a left injection pot 05-1, and a right injection pot 05-2. The left injection pot 05-1 can connect to the frame 02 through the left tube line 04-1 and the side holes 02-3. The frame 02, the cover film 03, the right injection pot 05-2 and the right tube line 04-2 can form an integrated structure and create a closed chamber 20 over a wound.

When the SACBD covers a wound, the left injection pot 05-1, the left tube line 04-1 and the side holes 02-3 can be used as an injection end pathway configured to inject artificial amniotic fluid and/or additives into the closed chamber 20, and the right injection pot 05-2, the right tube line 04-2 and the top hole 03-2 can be used as a drain end pathway configured to remove the artificial amniotic fluid and/or additives from the closed chamber 20, so that artificial amniotic fluid and/or additives can achieve an effective unidirectional cyclic flow from the injection end to the drain end, thus improving the wound washing efficiency.

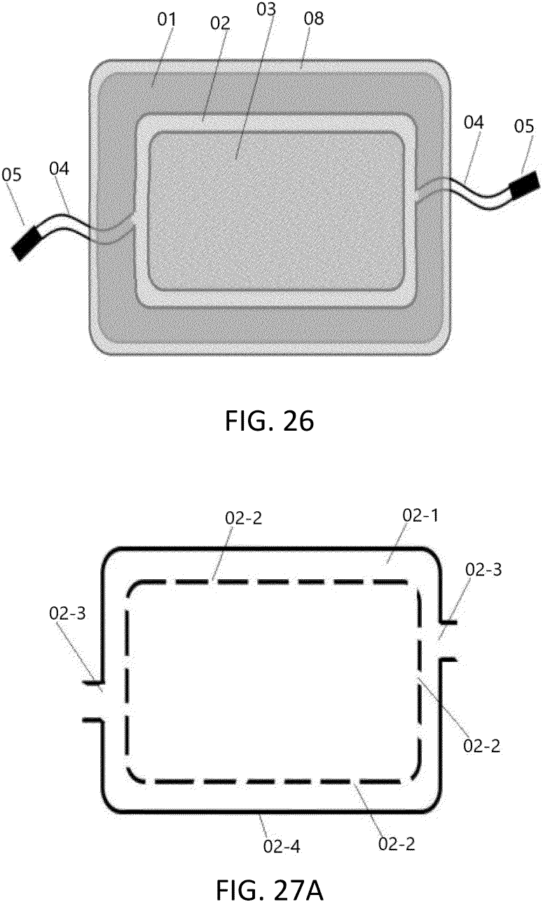

As shown in FIG. 26, according to the preferred embodiment of the present application, the SACBD can comprise a fixation membrane 01, a frame 02, a cover film 03, a tube line 04, and an injection pot 05, a protective film 08. The protective film 08 can be a smooth and transparent plastic film, which shape and size can be consistent with or slightly beyond an outer edge of the fixation membrane 01. The fixation membrane 01, the frame 02 and the cover film 03 can form an integrated structure, which can be covered with the protective film 08, so as to avoid being contaminated by external microorganisms.

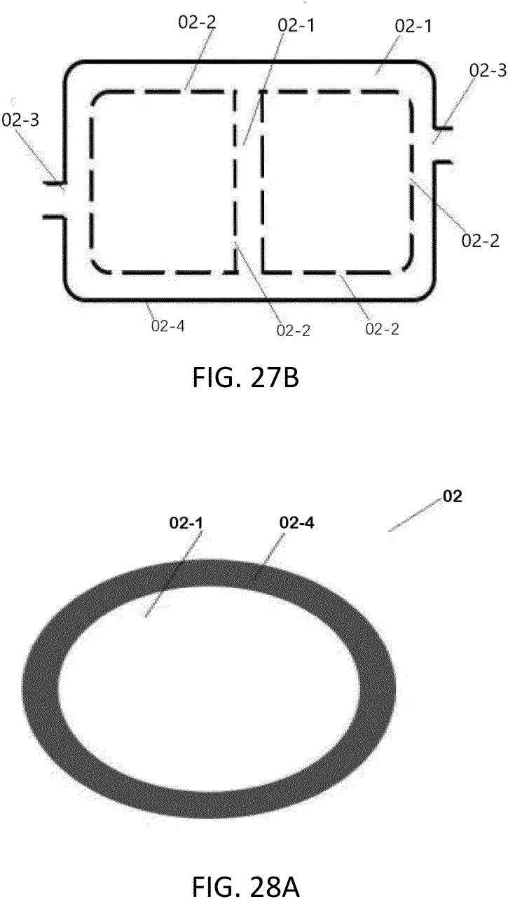

Referring to FIGS. 27A to 29, according to the preferred embodiment of the present application, a frame 02 of a SACBD can comprise a cavity canal 02-1, a plurality of micropores 02-2, a plurality of side holes 02-3, and a frame body 02-4. The frame body 02-4 can be as a supporting structure of the SACBD, and as an outer wall structure of the cavity canal 02-1. The cavity canal 02-1 can be set within the frame body 02-4 or outside the frame body 02-4, which can include a plurality of micropores 02-2 and at least one side hole 02-3.

As shown in FIGS. 27A and 32, according to the preferred embodiment of the present application, a frame 02 of the SACBD can comprise a cavity canal 02-1, a plurality of micropores 02-2, a plurality of side holes 02-3, and a frame body 02-4. The cavity canal 02-1 can include a plurality of micropores 02-2 and two side holes 02-3, which can be set inside the frame 02-4. The frame 02 can be usually made of transparent plastic material, or other medical polymer materials. The cavity canal 02-1 usually has an inner diameter no more than 5 mm, preferably from 0.5 mm to 2.5 mm. The micropores 02-2 can be scattered in an inner edge of the cavity canal 02-1, one end of the micropore 02-2 can connect the cavity tube 02-1, and the other end can connect to a closed chamber 20 composed of a cover film 03, the frame 02 and a wound. The micropores 02-2 can be usually with an aperture no more than 0.5 mm, preferably from 0.05 mm to 0.25 mm, with a gap no more than 20 mm, preferably from 5 mm to 10 mm. The two side holes 02-3 can be dispensed separately on an outer edge of the cavity line 02-1 or on an upper edge of the cavity line 02-1, which can connect with a tube line 04 with an aperture no more than 2.5 mm, preferably from 0.5 mm to 1.0 mm.

In addition, as shown in FIGS. 27B and 32, according to the preferred embodiment of the present application, a frame 02 of the SACBD can comprise a plurality of branch frames 02, which can be set between the frame 02. The branch frame 02 can include a cavity canal 02-1, a plurality of micropores 02-2, and a frame body 02-4. The branch frame 02 can be usually set in a larger size SACBD configured to support the larger size SACBD and prevent from compressing a wound, moreover, an efficiency of injecting or draining artificial amniotic fluid can be improved with an aid of the cavity canal 02-1 and micropores 02-2 of the branched frames 02.

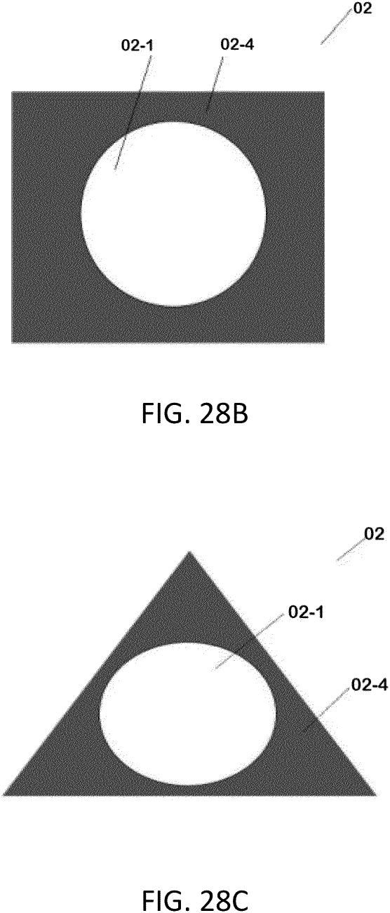

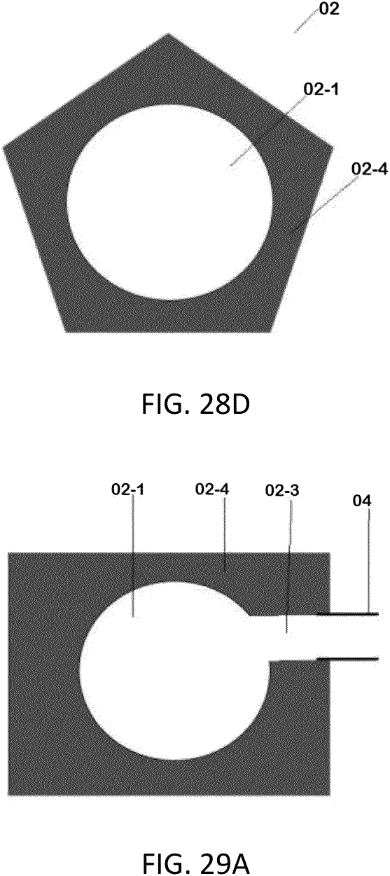

According to the preferred embodiment of the present application, a frame 02 of the SACBD can be set up in a different profile or size based on a wound with a variety of structural types. As shown in FIG. 28A, the frame 02 can be a tubular structure in cross section, including a cavity canal 02-1 and a frame body 02-4, which tube wall can be a part of the frame body 02-4, thus simplifying the frame 02 structure and reducing manufacturing cost of the SACBD. As shown in FIG. 28B, a frame 02 can be a columnar structure in cross section, including a cavity canal 02-1 and a frame body 02-4, and the frame body 02-4 can enhance a robot support for the SACBD configured to cover a larger area wound. As shown in FIG. 28C, a frame 02 can be a triangular cone structure in cross section, including a cavity canal 02-1 and a frame body 02-4, and the frame body 02-4 can enhance a robust pressure resistance for the SACBD. As shown in FIG. 28D, a frame 02 can be a multilamellar columnar structure in cross section, including a cavitary duct 02-1 and a frame 02-4, which can enhance a robust support and pressure resistance for the SACBD with less material when manufactured.

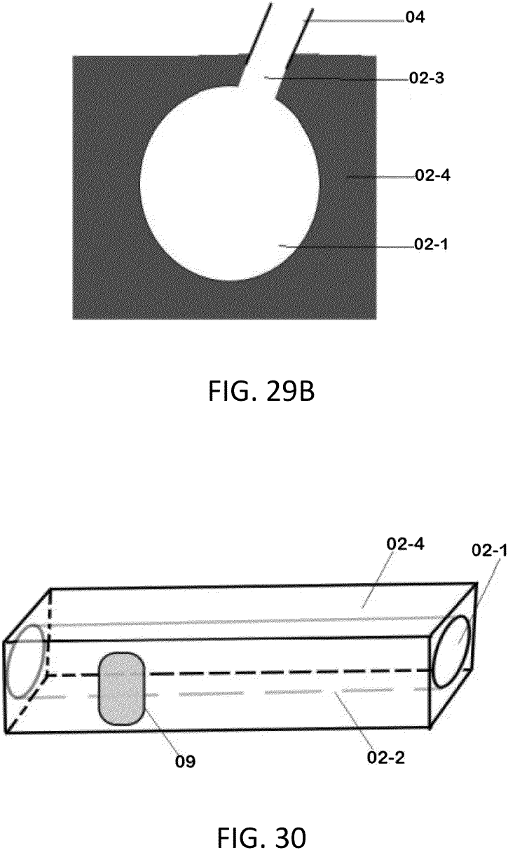

According to the preferred embodiemntof the present application, a side hole 02-3 of a frame 02 can be distributed at an outer edge or upper side edge of a cavity canal 02-1. As shown in FIG. 29A, a side hole 02-3 of a frame 02 can be set at an outer edge of a cavity canal 02-1, which can connect with a tube line 04 through the side hole 02-3 and the frame body 02-4. As shown in FIG. 29B, a side hole 02-3 of a frame 02 can be set at an upper side edge of a cavity canal 02-1, and the side hole 02-3 can connect with a tube line 04 through the frame body 02-4.

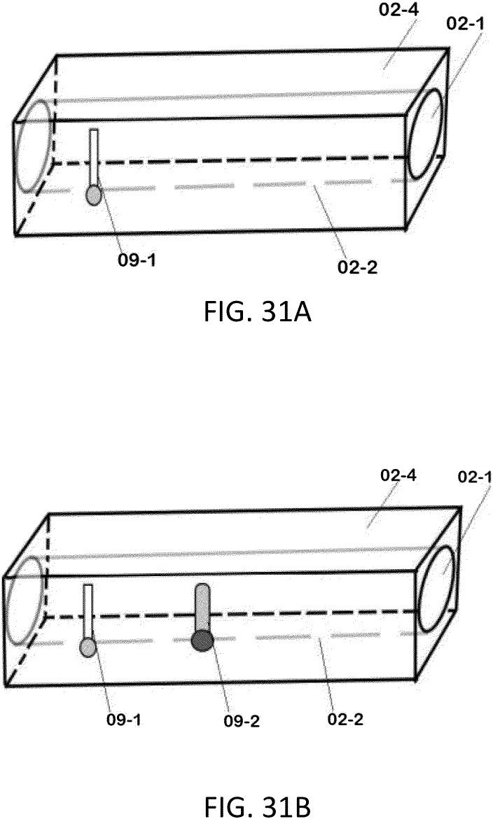

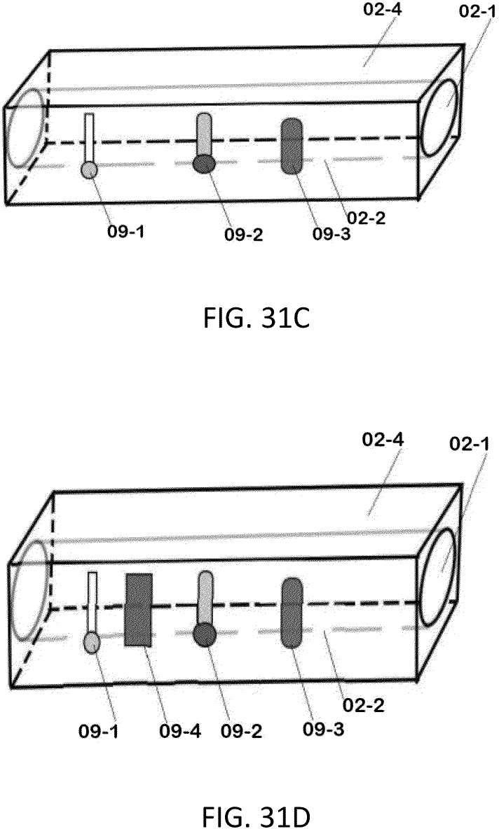

A SACBD can configured to provide a warm, tightly closed, moist, slightly acid, sterile healing microenvironment for a wound, which effect can be inspected with an aid of monitoring tools. Referring to FIGS. 30 and 31A to 31D, according to an example of the present application the SACBD can comprise a frame 02 and a display component 09.

As shown in FIGS. 30, 31A to 31D and 32, a frame body 02-4 of a frame 02 can be a columnar structure with a cavity canal 02-1, and a display component 09 can be set up at an internal wall surface of the cavity canal 02-1 in a detachable connection manner. The display component 09 can include a pH indicating unit 09-3, a temperature indicating unit 09-1, a humidity indicating unit 09-2, and/or a microbial indicating unit 09-4 configured to detect pH, temperature, humidity and microbial indicators of artificial amniotic fluid in a closed chamber 20 between the SACBD and a wound, respectively.

In addition, as shown in FIGS. 31A and 32, according to the preferred embodiment of the present application, a display component 09 of the SACBD can comprise a temperature indicating unit 09-1 configured to detect a temperature of artificial amniotic fluid in a closed chamber 20. The temperature indicating unit 09-1 can choose a miniature safe kerosene thermometer with a measure range from 28° C. to 40° C. The temperature of artificial amniotic fluid in a closed chamber 20 can be maintained at 36.5° C. to 39.5° C. as appropriate, if the temperature falls below 36.5° C., a heat preservation can be required, and if the temperature increase above 39.5° C., a wound inflammation might appear, a prompt replacement of fresh artificial amniotic fluid can be required, and/or sensitive antibiotics can be added if necessary.

As shown in as shown in FIGS. 31B and 32, according to the preferred embodiment of the present application, a display component 09 of the SACBD can comprise a temperature indicating unit 09-1 and a humidity indicator unit 09-2 configured to detect a temperature and humidity of artificial amniotic fluid in a closed chamber 20. The humidity indicating unit 09-2 can be a micro hygrometer with a measure range from 80% to 100%. The humidity of artificial amniotic fluid in a closed chamber 20 can be 100% as appropriate, if the humidity is less than 100%, the SACBD should be inspected dressing firstly, and adhesions might be reinforced if there is a leak.

As shown in as shown in FIGS. 31C and 32, according to the preferred embodient of the present application, a display component 09 of the SACBD can comprise a temperature indicating unit 09-1, a humidity indicator unit 09-2, and a pH indicator unit 09-3 configured to detect a temperature, humidity and pH of artificial amniotic fluid in a closed chamber 20 respectively. The pH indicator unit 09-3 can be selected with a miniature pH detection rod, which includes a methyl red indicator core, and a clear shell, the methyl red indicator core can be placed into the clear shell. The methyl red indicator core can include a methyl red indicator and a fiber rod. The clear shell can include a plurality of tiny pores configured for liquid penetration of the methyl red indicator core, while avoiding excessive liquid penetration and reducing the methyl red indicator detection effect. The methyl red indicator discoloration can range from pH4.2 to pH6.3. The maintenance of artificial amniotic fluid pH value from pH4.0 to pH6.0 in a closed chamber 20 can be optimal, and if the artificial amniotic fluid pH value is lower than 4.0 or higher than 6.0 in the closed chamber 20, the artificial amniotic fluid should be replaced.

As shown in as shown in FIGS. 31D and 32, according to the preferred embodiemnt of the present application, a display component 09 of the SACBD can comprise a temperature indicating unit 09-1, a humidity indicator unit 09-2, a pH indicator unit 09-3 and a microorganism indicator unit 09-4 configured to detect a temperature, humidity, pH and microorganism of artificial amniotic fluid in a closed chamber 20 respectively.

The microorganism indicator unit 09-4 can be a micro microbial detection kit that includes a plurality of micro test strips and a clear box, and the micro test strips can be arranged in parallel or serially within the clear box. The micro test strips can include a Gram-negative bacteria test strip, a Gram-positive bacteria test strip, and a fungi test strip. The clear box can comprise a plurality of tiny pores for liquid infiltration. If the micro test strip shows a positive result, a category of wound infection can be preliminarily judged, then artificial amniotic fluid can need to be replaced, and a sensitive antibiotic would be added if necessary.

It should be illustrated that a combination modality of display component 09 of the SACBD can be selected depending on a wound type. Such as a sterile surgical incision, the SACBD can be without the display component 09, or with a simply display component 09 including a temperature indicating unit 09-1, a humidity indicating unit 09-2, and a pH indicating unit 09-3. Such as a contaminated surgical incision or traumatic wound, the display component 09 of a SACBD can include a temperature indicator unit 09-1, a humidity indicator unit 09-2, a pH indicator unit 09-3, and a microorganism indicator unit 09-4.

Referring to FIGS. 22 to 24 and 30 to 35, according to the preferred embodiment of the present application, a method 400 of the SACBD can comprise the following steps.

S410. Select a fabricated SACBD according to a wound shape and size. The fabricated SACBD can include a plurality of models with a different type of shapes and functional combinations configured to cover the wound. For example, the fabricated SACBD can include a dressing main body and a plurality of fittings, as shown in FIGS. 22 to 24, the fittings can include an injection pot 05, a three-way 06, and a plurality of valves 07, or as shown in FIGS. 32 to 35, the fabricated SACBD can include a dressing main body, a plurality of fittings, and a plurality of components, the components can include a syringe 30, an exhaust needle 40, an infusion bag 50, and a urine collection bag 60, or as shown in FIGS. 30 and 31A to 31D, the components can include a temperature indicating unit 09-1, a humidity indicating unit 09-2, a pH indicating unit 09-3, and a microorganism indicating unit 09-4.

Additionally, the SACBD can be fabricated bedside, which can include a plurality of steps as follow: (1) evaluating a wound shape and size, (2) cutting a fixation membrane, a frame and a cover film based on the wound shape and size, (3) adhesive splicing the fixation membrane, the frame and the cover film as an all-in-one construct, (4) bonding a pH indicating unit and/or temperature indicating unit and/or humidity indicating unit and/or microorganism indicating unit with the frame, (5) connecting a plurality of fittings with the all-in-one construct.

S120. Adhesively fix the SACBD on the wound. As shown in FIGS. 32 and 33, the frame 02 of the SACBD can completely cover the wound W, a tube line 04-1 at an injection end can be usually set at an upper end or proximal end of the wound W, and a tube line 04-2 at a drainage end can be set at a lower end or distal end of the wound W, then the fixation membrane can be fixed to health skin around the wound W, and then checking a tightness of a closed chamber 20 being created with the SACBD and the wound W using a syringe 30 to suction injection pot 05-1 or injection pot 05-2, when an air leakage being observed, checking whether or not the SACBD broken, and checking whether or not the fixation membrane 01 sticking reliable, if necessary, reinforcing with an infusion patch. In addition, if being used in an annular wound W with a larger area of an extremity, it is necessary to avoid taping of the fixation membrane 01 to the wound W.

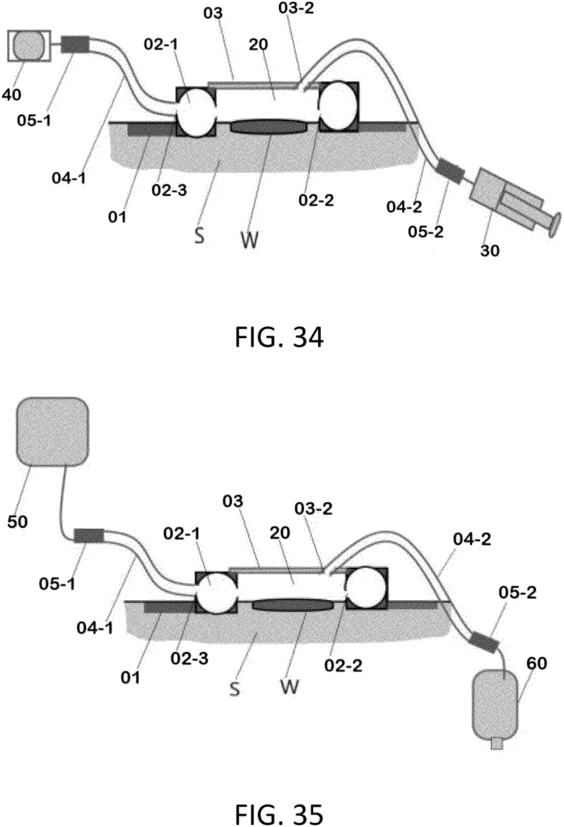

S130. Supplement with artificial amniotic fluid and additives into the closed chamber 20. As shown in FIGS. 33 and 34, firstly, loading the artificial amniotic fluid and additives into the syringe 30, connecting the syringe 30 to the injection pot 05-1, and connecting an exhaust needle 40 to the injection pot 05-2, and then pushing the syringe 30, the artificial amniotic fluid and additives flowing through a tube line 04-1, a plurality of side holes 02-3, a cavity canal 02-1, and a plurality of micropores 02-2 into the closed chamber 20, and air inside the closed chamber 20 being discharged through a top hole 03-2, a tube line 04-2, the injection pot 05-2, and the exhaust needle 40, until the closed chamber 20 being filled with the artificial amniotic fluid and additives, and then, checking sealing of the closed chamber 20, and displaying values of a pH and/or temperature and/or humidity and/or microorganism in real time manner. Usually, the SACBD can label with a rated capacity configured to guide a configuration of the artificial amniotic fluid and additives.

When a wound area is larger, a large-size SACBD is needed, or a continuously wound surface irrigate is needed, such as an obvious contamination or suspicious infection wound, can be adopted a scheme as shown in FIG. 35. Firstly, the artificial amniotic fluid and additives can be loaded into an infusion bag 50 which can be suspended from an infusion rack or coupled with an infusion pump, then the infusion bag 50 can be connected to the injection pot 05-1, and a collection bag 60 being placed in a low position can be connected to the injection pot 05-2, and then the artificial amniotic fluid and additives can enter into the closed chamber 20 through the tube line 04-1, the side holes 02-3, the cavity canal 02-1 and the micropores 02-2, finally, dispose of artificial amniotic fluid and additives in the closed chamber 20 can flow into the collection bag 60 through a top hole 03-2 and a tube line 04-2 configured to replace the artificial amniotic fluid and additives and/or irrigate the wound.

It was noted that the artificial amniotic fluid component can be an isotonic balanced salt solution containing phosphate or carbonate buffer with a pH from 3.5 to 5.5, and the additives can comprise a plurality of antibiotic, a plurality of protease, a plurality of growth factors, a plurality of stem cells, a plurality of hemostatic agents, a plurality of pain relief agents, etc.

S140. Observe the wound healing progress regularly. As shown in FIGS. 30, 31A to 30D and 32, the wound can be observed through the cover film 03, and the wound healing situation as well as values of the pH and/or temperature and/or humidity and/or microorganism can be judged on-site or remotely by a medical staff.

If a temperature displayed by the temperature indicating unit 09-1 falls below 36.5° C., a heat preservation can be required, and if the temperature is above 39.5 °Cmight herald a presence of inflammation in the wound, a prompt replacement of the artificial amniotic fluid can be required, and/or sensitive antibiotics can be added.

If the pH indicating unit 09-3 shows a pH value below 4.0 or above 6.0, the artificial amniotic fluid can be replaced promptly to ensure the wound with a pH microenvironment between 4.0 and 5.5.

If the microorganism indicator unit 09-4 shows positive results, it can be preliminarily judged that the wound infection category, such as the wound infection possibly due to a Gram-negative bacteria or/and a Gram-positive bacteria or/and a fungi, and fresh artificial amniotic fluid and/or a sensitive antibiotic can be needed.

When a replacement of artificial amniotic fluid is required, as shown in FIG. 34, an exhaust needle 40 can connect an injection pot 05-1, and a syringe 30 can connect an injection pot 05-2, then pulls the syringe 30 and draws out some or all of the artificial amniotic fluid and additives.

S150. Remove the SACBD when the wound healed.

Examples of the present application mentioned above does not place any limitation on a technical scope of the application, any slight modifications, equivalent changes and modifications made to the above examples by the technical essence of the application remain within the scope of the technical scheme of the application. Professionals should be aware that a skilled person may use different methods for each particular application to achieve the described function, but such implementation should not be considered beyond the scope of this application.

What is claimed is:

1. An intelligent biomimetic dressing providing a warm, moist, isolated, slightly acid, pressure free and sterile microenvironment for a wound, comprising:
an outer layer configured to insulate the wound from air; and
an interlayer configured to provide a warm, humid, slightly acidic and sterile microenvironment, the interlayer including:
a microfluidic system, and
an inner layer configured to support the interlayer and the outer layer to construct a closed chamber on the wound.

2. The intelligent biomimetic dressing according to claim 1, wherein the inner layer comprises a framework configured to avoid direct contact of the interlayer with the wound.

3. The intelligent biomimetic dressing according to claim 2, wherein the interlayer comprises a plurality of lamp tubes configured to emit infrared and/or far infrared and/or ultraviolet light for treating the wound.

4. The intelligent biomimetic dressing according to claim 3, wherein the interlayer further comprises a monitoring unit configured to collect wound microenvironment data, and the monitoring unit includes a plurality of sensors which connect with a power supply and a control unit.

5. The intelligent biomimetic dressing according to claim 4, wherein the plurality of sensors comprises a combination of one or more of temperature sensor, humidity sensor, oxygen saturation sensor, pressure sensor, pH sensor, and microbial sensor.

6. The intelligent biomimetic dressing according to claim 1, further comprising:
a filling unit configured to add artificial amniotic fluid to the microfluidic system.

7. The intelligent biomimetic dressing according to claim 6, wherein the microfluidic system comprises a reservoir, a first micropump, a microvalve, and a connecting tube, the connecting tube connects with the microfluidic system, and the first micropump electrically connects with a power supply and a control unit.

8. The intelligent biomimetic dressing according to claim 7, wherein the reservoir comprises an injection pot configured to connect a syringe and a capsular bag configured to store artificial amniotic fluid and/or additives.

9. The intelligent biomimetic dressing according to claim 1, further comprising:
a heating unit configured to provide a warm environment for the wound, and the heating unit electrically connects with a power supply and a control unit.

10. The intelligent biomimetic dressing according to claim 9, further comprising:
a control unit configured to process data and manipulate a micropump, a smart valve, and a heating component, and the control unit electrically connects with an intelligent terminal, a power supply, a sensor, the micropump, the smart valve, and the heating component.

11. The intelligent biomimetic dressing according to claim 10, further comprising:
a waste liquid collection unit configured to pool waste liquid, the waste liquid collection unit includes a connecting tube, a smart valve, a second micropump, and a waste sac, the second micropump and the smart valve electrically connect to a power supply and a control unit, and the second micropump and the smart valve connect to a microfluidic system through the connecting tube.

12. The intelligent biomimetic dressing according to claim 11, wherein the microfluidic system comprises a first port, a capillary net, a second port, and an end port, the first port connects with a reservoir and the capillary net, the second port scatters in the capillary net, the capillary net connects with an inner layer through the second port, and the end port connects with the waste sac.

13. The intelligent biomimetic dressing according to claim 1, further comprising:
a plurality of combinations configured to cover a plurality of wounds, and the combination includes an outer layer, an interlayer and an inner layer, which shares a filling unit, a power supply, a waste liquid collection unit, and a control unit.

14. A simulated amniotic cavity biomimetic dressing for providing a closed, moist, slightly acidic and sterile microenvironment to a wound, comprising:
a dressing body configured to cover the wound and create a closed chamber, the dressing body includes a covering film, a frame, and a fixation membrane, the covering film covers the frame, and the fixation membrane adheres to the frame, and the frame includes a cavity canal configured to flow artificial amniotic fluid, and
a plurality of fittings configured to inject the artificial amniotic fluid into the closed chamber and drain waste liquid, and the fittings include a plurality of tube lines, an injection pot, and a valve.

15. The simulated amniotic cavity biomimetic dressing according to claim 14, wherein the covering film, the frame and the fixation membrane are combined into an integrated structure configured to cover the wound to create the closed chamber.

16. The simulated amniotic cavity biomimetic dressing according to claim 14, further comprising:
an accessory configured to collect environmental data within the closed chamber, and the accessory includes a pH indicating unit, a temperature indicating unit, a humidity indicating unit, and a microbial indicating unit.

17. The simulated amniotic cavity biomimetic dressing according to claim 16, wherein the microbial indicating unit comprises a plurality of micro test strips and a clear box, and the micro test strips include a Gram-negative bacteria strip, a Gram-positive bacteria strip, and a fungi strip.

18. The simulated amniotic cavity biomimetic dressing according to claim 14, wherein the cavity canal comprises:
a plurality of micro holes configured to communicate the closed chamber, and
a plurality of side holes configured to connect the tube lines.

19. The simulated amniotic cavity biomimetic dressing according to claim 18, wherein the tube line comprises:
an inlet tube configured to inject artificial amniotic fluid into the closed chamber, and
a drain tube configured to drain waste liquid from the closed chamber.

20. A method of a biomimetic dressing, comprising:
selecting a biomimetic dressing selected form a group consisting of an intelligent biomimetic dressing and a simulated amniotic cavity biomimetic dressing,
covering a wound with the biomimetic dressing to create a closed chamber,
injecting at least one of an artificial amniotic fluid and additives into the closed chamber, and
providing a warm, moist, slightly acidic, isolated, unstressed, and sterile microenvironment to the wound.
